(19) United States
(12) Patent Application Publication
(10) Pub. No.: US 2009/0211574 A1
(43) Pub. Date: Aug. 27, 2009

(54) Airway device and methods of making and use thereof

(76) Inventor: Joseph A. Sniadach, Baltimore, MD (US)

Correspondence Address: J. Bruce Hoofnagle, P.O. Box 370, Lisbon, MD, 21765-0370, US

(21) Appl. No.: 12/074,736

(22) Filed: Mar. 6, 2008

Related U.S. Application Data
(60) Provisional application No. 61/066,567, filed on Feb. 21, 2008.

(51) Int. Cl. A61M 16/04; A62B 18/02

(52) U.S. Cl. 128/200.26; 128/207.14; 128/206.21

(57) ABSTRACT

An airway device 64 includes a hollow tube 51 having a proximal end 65b and a distal end 71, and is formed with a straight section 65 and a curved section 68. The tube 51 can include a proximal portion 65a, which extends from the proximal end 65b for receipt of a face mask 180. When the airway device 64 is placed in a patient's mouth 28, oral cavity 34 and oropharyngeal area 36, the distal end 71 is located at the patient's glottic opening 46 and vocal cords 40. Inflatable cuffs 75, 73 and 73a may be located individually on the tube 51 in selectable single, double and triple cuff embodiments to form seals in the areas of the patient's mouth 28, oral cavity 34, oropharyngeal area 36, glottic opening 46 and vocal cords 40, esophageal opening 48 and inner nasal-passage opening 33.

This application claims the benefit of U.S. Provisional Application No. 61/066,567, filed Feb. 21, 2008, which is incorporated herein by reference thereto.

### BACKGROUND OF THE INVENTION

This invention relates to an airway device, and methods of making and use thereof, for ventilating a medical patient. In particular, this invention relates to an airway device, and methods of making and use thereof, which includes a single-cuffed, or a multiple-cuffed, oral airway, and which may include a mask combined therewith.

Basic cardiopulmonary resuscitation begins with the establishment and maintenance of an adequate airway. Different designs of airway devices are used in an effort to achieve a patient airway and provide ventilation through oxygenation and removal of expired carbon dioxide. Furthermore, some currently used airway devices attempt to achieve a second airway with an endotracheal tube.

Due to the problem of many patients exhibiting a difficult intubation or difficult ventilation, the medical field has continued to search for an airway device that achieves adequate ventilation, especially in a difficult airway scenario. Further, if the airway device, which achieves improved ventilation, also acts as a conduit for endotracheal intubation, the oropharyngeal manipulation is minimized with the decreased upper airway edema, suctions, and possible bleeding. Inability to achieve ventilation, especially when hampered by bleeding and suctions, ultimately leads to a surgically-obtained airway. An effective airway device must also be easy to use with a quick learning curve.

The American Red Cross CPR courses and Advanced Cardiac Life Support teach adequate ventilation and oxygenation as life dependent maneuvers. According to teachings in the medical literature, Bag Mask Ventilation is the most basic airway skill to master. Yet, it is often ineffective, either due to the patient's anatomy or inexperienced operators. As noted in paramedical literature, there has been a recent evaluation of patient ventilation and prehospital intubation, the reports from which indicate that ventilation still has not been adequately mastered, and that many patients have died from misplaced endotracheal tubes in the esophagus.

Although the endotracheal tube provides a definitive airway that is secured, it is lethal when performed improperly. Prehospital providers with limited experience and varying skills, when placed in uncontrolled environments and emergency situations, are at high risk of improper placement of an endotracheal tube. Mask ventilation provides life sustaining oxygenation as a primary skill or as a back-up when tracheal intubation fails. The aim of the airway device of this invention is to provide ventilation and improved oxygenation due to its shape and cuffs, with or without a mask combination, as well as with or without an endotracheal tube conduit.

Many airway devices are currently used with marginal results and, although such devices are used with a mask, they are not physically a part of the mask complex, which allows for potential operator error and inadequate ventilation. Mask ventilation is the most basic form of ventilation, yet it remains one of the most undermastered. Although endotracheal intubation is considered the definitive airway, which secures the airway, the misplacement of endotracheal tubes still occur on a daily basis, and serve as a potential death sentence. Recent comments in paramedic literature indicate that lives saved by endotracheal tubes are offset by lives lost from endotracheal intubation. There is a call in such literature for a refocus on mask ventilation and for it to be performed effectively.

In a definitive technique of ventilating the patient, an endotracheal tube is usually placed within the patient's mouth by direct laryngoscopy, where the epiglottis is lifted off of the glottic opening for placement of the distal end of the tube through the glottic opening, stenting it open, and displacing the anterior of the epiglottis. Many times, particularly in emergency and out-of-hospital situations, the use of the endotracheal tube and the laryngoscopy process is not available. In such situations, the care giver has to resort to using a conventional airway device.

In one example of such a conventional airway device, a distal end of the device falls short of extending to the glottic opening of the patient. In an effort to overcome this shortcoming, an inflatable cuff was added to the device in an attempt to prevent undesirable backflow of air through the patient's mouth. However, the conventional airway device, with or without the inflatable cuff, is not locatable within the proximity of, or at, the glottic opening, and does not extend sufficiently to contact the epiglottis. In fact, if such a conventional airway is placed in the patient by inexperienced medical personnel, they may push the patient's tongue downward against the epiglottis, forcing it to overlie the trachea, making ventilation of the patient difficult, if not impossible.

This problem of difficult ventilation of the patient occurs very frequently followed by a lack of oxygen and patient injury, if not immediately corrected. Blood, secretions, airway edema or a difficult anatomic airway may make initial endotracheal intubation or re-intubation impossible.

### SUMMARY OF THE INVENTION

It is an object of this invention to provide an airway device for achieving patient ventilation and improving patient oxygenation with structural features, which are highly effective in emergency conditions, as well as controlled environments. The airway device of this invention is designed to achieve improved ventilation of the patient over currently available devices.

Another object of this invention is to provide an airway device which not only clearly enhances and improves the application of patient ventilation in the controlled setting of the operating room, but provides the ability to be used quickly and effectively by prehospital personnel, in emergency situations.

With these and other objects in mind, this invention contemplates an airway device having a tube formed with a prescribed length which facilitates the location of a distal end of the tube at a patient's glottic opening and vocal cords.

In addition, this invention contemplates an airway device having a tube with a proximal portion which extends from a proximal end of the tube by a prescribe distance, when the tube is in a patient's oral cavity and oropharyngeal area.

Further, this invention contemplates an airway device having a tube with one or more inflatable cuffs mounted on the tube for providing seals about various locations within the patient's oral cavity and oropharyngeal area.

Still further, this invention contemplates an airway device having a multiple-piece tube for facilitating the formation of a proximal portion which extends from a proximal end of the tube by a prescribe distance, when the tube is in a patient's oral cavity and oropharyngeal area.

Additionally, this invention contemplates an airway device having a tube with a distal end positionable at a patient's glottic opening, with the distal end being bevelled at various angles.

Other objects, features and advantages of the present invention will become more fully apparent from the following detailed description of the preferred embodiment, the appended claims and the accompanying drawings.

### BRIEF DESCRIPTION OF THE DRAWINGS

In the accompanying drawings:

FIG. 1 is a break-away side view showing a patient's head to show the relative locations of various elements of an airway anatomy of the patient;

FIG. 2 is a side view of showing first prior art airway device;

FIG. 3 is a side view showing a second prior art airway device;

FIG. 4 is a side view showing a first embodiment of an airway device in accordance with certain principles of the invention;

FIG. 5 is a side view showing a second embodiment of the airway device in accordance with certain principles of the invention;

FIG. 6 is a side view showing a third embodiment of the airway device in accordance with certain principles of the invention;

FIG. 7 is a side view showing a fourth embodiment of the airway device in accordance with certain principles of the invention;

FIG. 8 is a side view showing a fifth embodiment of the airway device in accordance with certain principles of the invention;

FIG. 9 is a side view showing a sixth embodiment of the airway device in accordance with certain principles of the invention;

FIG. 10 is a side view showing the first embodiment of the airway device of FIG. 4, in assembly with the patient of FIG. 1, in accordance with certain principles of the invention;

FIG. 11 is a partial section view showing a mask for use with the first embodiment of the airway device of FIG. 1 in accordance with certain principles of the invention;

FIG. 12 is a side view showing the second embodiment of the airway device of FIG. 5, in assembly with the patient of FIG. 1, in accordance with certain principles of the invention;

FIG. 13 is a side view showing the third embodiment of the airway device of FIG. 6, in assembly with the patient of FIG. 1, in accordance with certain principles of the invention;

FIG. 14 is a side view showing the fourth embodiment of the airway device of FIG. 7, in assembly with the patient of FIG. 1, in accordance with certain principles of the invention;

FIGS. 15 through 20 are partial side views showing six different structural designs for a distal end of an airway device, where the distal end of each of the first through the sixth embodiments of the airway devices of FIGS. 4 through 9, respectively, could be formed optionally with any of the six different designs, in accordance with certain principles of the invention;

FIG. 21 is a side view showing an airway device in assembly with the patient of FIG. 1, positioned for receipt of a stylette used for guiding an intubation tube into ventilating assembly with the patient, in accordance with certain principles of the invention; and

FIG. 22 is a side view showing an airway device in assembly with the patient of FIG. 1, and includes facility for providing an air-blockage seal during the ventilating of the patient, in accordance with certain principles of the invention.

### DESCRIPTION OF THE PREFERRED EMBODIMENTS OF THE INVENTION

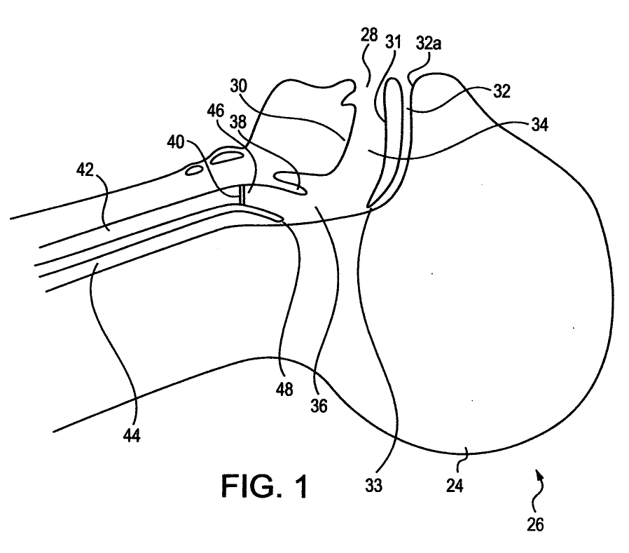

As shown in FIG. 1, a head 24 of a patient 26 is oriented to illustrate the relative locations of the patient's mouth 28, tongue 30, palate 31, nasal cavity 32, outer nasal cavity opening 32a, inner nasal-cavity opening 33, oral cavity 34, oropharyngeal cavity 36, epiglottis 38, vocal cords 40, trachea 42, esophagus 44, glottic opening 46, and outer esophageal opening 48.

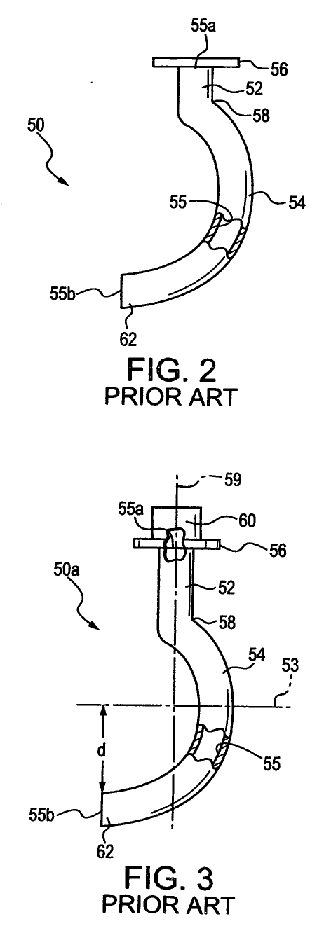

Referring to FIGS. 2 and 3, a pair of prior art hollow airway devices 50 and 50a are each formed with a straight proximal section 52 and a curved distal section 54. Each of the devices 50 and 50a is formed with a hollow passage 55 extending between, and including, a proximal opening 55a and a distal opening 55b, at respective ends of the devices. In each of the devices 50 and 50a, the length of the straight section 52 is referred to as a limited peri-lingular length, which is defined as the distance between a flange 56 and a juncture 58 of the straight section with the curved section 54. The airway device 50a (FIG. 3) is also formed with an adapter 60 for connection of the airway device to an external ventilating facility.

With the straight section 52 of each of the prior art devices 50 and 50a being formed with the limited peri-lingular length, the straight section 52 of each of the prior art devices will be located totally within the patient's oral cavity 34, when the devices are used to provide an airway for the patient 26. With the straight section 52 being located within the patient's oral cavity 34, the flange 56 and the adapter 60 (FIG. 3) will be located immediately outside of the patient's mouth 28.

The curved section 54 of each of the hollow devices 50 and 50a extends, for a limited axially-curvilinear distance, from the juncture 58 to a distal end 62 of each of the devices, at which the distal opening 55b is located. With the curved section 54 extending for the above-noted limited axially-curvilinear distance only, neither the distal end 62, nor the distal opening 55b, will extend to, or beyond, a free-end tip of the patient's epiglottis 38, in a direction away from the patient's mouth 28. In fact, when either of the airway devices 50 and 50a is used to ventilate the patient 26, the distal end 62 and the distal opening 55b of the hollow passage 55 would be spaced considerably, toward the mouth 28, in a direction away from the vocal cords 40, the glottic opening 46, and the epiglottis 38.

In addition, as shown in FIG. 3, due to a relatively sharp, slightly obtuse, angle of departure by the curved section 54 from the straight section 52, at the juncture 58, a major portion of the curved section 54 extends fully beyond, and is completely spaced from, one side (i.e., to the right) of a centerline 59, or central axis, of the straight section 52. With this structural arrangement, a minor portion of the curved section 54, which extends from the major portion thereof to the distal end 62, is located on the other side (i.e., to the left) of the centerline 59. With the major portion of the curved section 54, of the device 50a, being located to the one side (i.e., to the right) of the centerline 59, and only a minor portion of the curved section being located to the other side (i.e., to the left) of the centerline, it is possible that difficulties could be encountered when an attempt is made to insert the device into a ventilating position within the patient 26.

As further shown in FIG. 3, the farthest extension of the above-noted major portion of the curved section 54, of the device 50a, which extends completely to the one side (i.e., to the right) of the centerline 59, is along a horizontal line 53, which is perpendicular to the centerline. With this fully extended curvature of the curved section 54 of the device 50a, the distal end 62 is spaced considerably, by a distance “d,” from one side of (i.e., below) the line 53. With this structural arrangement, only the very minor portion of the curved section 54 is located to the other side (i.e., to the left) of the centerline 59, and below the line 53. Again, with this structure, it is possible that difficulties could be encountered when an attempt is made to insert the device 50a into a ventilating position in the patient 26.

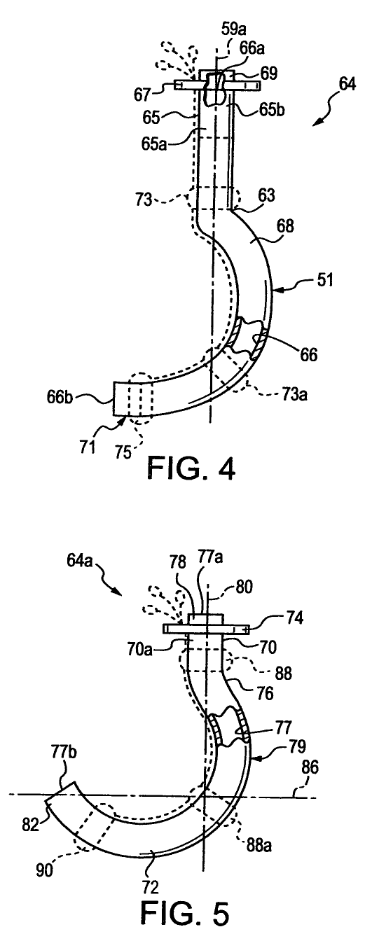

Referring to FIG. 4, in a first embodiment of an inventive airway device, an extended airway device 64 includes a tube 51 formed in an extended configuration. The tube 51 is formed with a hollow passage 66 extending between, and including, a proximal opening 66a of the passage, at a proximal end 65b of the tube, and a distal opening 66b of the passage, at an extended distal end 71 of the tube. The airway device 64 includes some features similar to those of the device 50a (FIG. 3), and further includes improvements embodied in certain principles of the invention of the airway device 64. Thus, the airway device 64 provides a device for those who wish to use an airway device having features similar to the device 50a, but with some of the advantages and benefits of the invention as described and set forth herein.

In one aspect of the invention, the tube 51 of the airway device 64 is formed with a straight section 65, about a centerline 59a, or central axis, having an extended peri-lingular length, which is longer than the limited peri-lingular length of each of the prior art devices 50 and 50a, shown in FIGS. 2 and 3, respectively. With the extended peri-lingular length, the straight section 65 of the tube 51 of the device 64 is formed with an extended proximal portion 65a, extending to the proximal opening 66a thereof. In addition, the tube 51 may be formed with a flange 67 and an adapter 69, at the proximal end 65b of the straight section 65, or, as described below, can be used, in accordance with certain principles of the invention, without the flange and the adapter.

In another aspect of the invention, independent of, or in conjunction with, the extended peri-lingular length of the straight section 65, the tube 51 can also be formed with a curved section 68, which is integrally joined with the straight section 65 at a juncture 63 of the straight and curved sections intermediate the proximal end 65b and the distal end 71 of the tube. The curved section 68 extends from the juncture 63 to the extended distal end 71 of the tube 51 of the device 64, at which the distal opening 66b is located. In this aspect of the invention, the axially curvilinear length of the curved section 68 from the juncture 63 is a prescribed extended curvilinear length, which is longer, from the juncture to the distal end 71, than the axially curvilinear length of the curved section 54, from the juncture 58 to the distal end 62, of each of the prior art airway devices 50 (FIG. 2) and 50a (FIG. 3).

Also, in another aspect of the invention, the distal end 71 of the tube 51 may be shaped in the form of a curvature such as, but not limited to, a continuation of the curvature of the curved section 68, as shown in FIG. 4, or may be formed as a straight section extending horizontally and distally from the curved section, without departing from the spirit and scope of the invention.

Further, in still another aspect of the invention, the straight section 65 of the tube 51 could be formed with the limited peri-lingular length only, as described above, in combination with the curved section 68, formed with the longer prescribed extended curvilinear length, and having the straight or curved distal end 71, also as described above, without departing from the spirit and scope of the invention.

The above-described structural features of the airway device 64 are distinctly different from the structural features of the prior art devices 50 (FIG. 2) and 50a (FIG. 3), and facilitate the establishment of a comparatively enhanced airway for the patient 26, when using the airway device 64 in the manner described below.

In another aspect of the invention, in a first single-cuff embodiment of the device 64, an inflatable distal cuff 75, which, in this embodiment of the device, is located at its distal position on the distal end 71 of the tube 51, spaced inboard from the distal opening 66b, as shown in FIG. 4. The distal cuff 75, which is the only cuff in this embodiment, is located on the curved section 68 of the tube 51 in such a manner that no portion of the cuff overlaps, or obstructs, the distal opening 66b.

In an additional aspect of the invention, in a second single-cuff embodiment of the device 64, an inflatable proximal cuff 73, which, in this embodiment of the device, is located at its proximal position on an intermediate portion of the straight section 65 of the tube 51, as shown in FIG. 4. The proximal cuff 73 is located inboard from the proximal opening 66b by a prescribed proximal distance. Further, the proximal cuff 73, at its proximal position, is the only cuff in this embodiment.

In a further aspect of the invention, in a first double-cuff embodiment of the device 64, the device includes only the distal cuff 75, in its distal position, and the proximal cuff 73, in its proximal position.

In another aspect of the invention, in a second double-cuff embodiment of the device 64, an intermediate cuff 73a is selectively located on the curved section 68 of the tube 51 between the proximal opening 66a and the distal cuff 75, at an intermediate location the tube as shown in FIG. 4. In this embodiment, the device 64 includes only the distal cuff 75, in its distal position, and the intermediate cuff 73a, in its intermediate position.

In still another aspect of the invention, in a triple-cuff embodiment of the airway device 64, the device includes the three inflatable cuffs 75, 73 and 73a assembled in their respective distal, proximal and intermediate positions on the tube 51, as illustrated in FIG. 4.

As shown in FIG. 5, in a second embodiment of an inventive airway device, a hook-shaped airway device 64a includes a tube 79 formed in a hook-shaped configuration. The tube 79 is formed with a hollow passage 77 extending between, and including, a proximal opening 77a of the passage, at a proximal end 70a of the tube, and a distal opening 77b of the passage, at a distal end 82 of the tube. The tube 79 of the airway device 64a is formed with a straight section 70, and a curved section 72. The straight section 70 extends from a flange 74 to a juncture 76 of the straight section with the curved section 72, with an adapter 78 extending outward from the flange.

Without departing from the spirit and scope of the invention, the length of the straight section 70, of the device 64a, could be consistent with the above-described limited peri-lingular length of the prior art devices 50 (FIG. 2) and 50a (FIG. 3), or the length of the straight section 70 could be consistent with the extended peri-lingular length of the device 64, as described above and as shown in FIG. 4, with or without the flange 74 and the adapter 78.

As further shown in FIG. 5, the curved section 72 of the tube 79 of the hook-shaped airway device 64a is formed generally in a semi-circular configuration, and extends from the juncture 76 to the distal end 82 of the device. The curved section 72 is integrally joined with the straight section 70 at the juncture 76 intermediate the proximal end 70a and the distal end 82 of the tube 79. The curved section 72 extends in a generally circular arc away from the juncture 76, and is located, partially, on one side (i.e., to the right) of a centerline 80 of the straight section 70. The curved section 72 continues in the generally circular arc, and partially, to an other side (i.e., to the left) of the centerline 80, which is opposite the above-noted one side thereof.

As noted above, and with respect to FIG. 3, due to the sharp, slightly obtuse, angle of departure of the curved section 54 from the straight section 52 of the device 50a, major portions of the curved section are located completely to the right of the centerline 59. In one aspect of the invention, and referring again to FIG. 5, the angular departure of the curved section 72 from the straight section 70, at the juncture 76, is formed at an obtuse angle, which is more obtuse than the above-noted sharp, slightly obtuse, angle of the device 50a (FIG. 3).

With the structure shown in FIG. 5, a first continuous portion of the curved section 72 extends between the juncture 76 and the distal end 82, and is located on the other side (i.e., to the left) of the centerline 80. Also, a second continuous portion of the curved section 72 extends from the juncture 76 toward, but not to, the distal end 82, and is located on the one side (i.e., to the right) of the centerline 80.

Thus, in FIG. 5, the portion of the curved section 72, of the tube 79, located in overlapping vicinity of the centerline 80, does not have any portions which are completely separated and spaced from the centerline. In comparison, as described above, a major portion of the curved section 54, of the device 50a (FIG. 3), which is in the vicinity of the centerline 59, is completely separated and spaced from overlapping arrangement with the centerline, resulting in a portion of the curved section being located fully to the one side (i.e., to the right in FIG. 3) of the centerline.

The curved section 72 continues in the circular arc and terminates at a distal end 82 thereof, which, in one aspect of the invention, is located a prescribed hooked curvilinear distance from the juncture 76. In addition, the distal end 82 of the curved section 72 extends the farthest to the other side (i.e., to the left) of the centerline 80, and is located along a horizontal line 86, which is perpendicular to the centerline.

It is noted that, as shown in FIG. 5, the farthest extension of the curved section 72 to the one side (i.e., to the right) of the centerline 80, and the farthest extension of the curved section 72 to the other side (i.e., to the left) of the centerline, are coincidentally located on the line 86. Further, the major curved portion of the curved section 72 is located on the other side (i.e., to the left) of the centerline 80, and below the line 86.

It is further noted that the curved section 72 could extend to a termination point, at the distal end 82, before, or after, its intersection with the line 86, without departing from the spirit and scope of the invention.

The above-described structural features of the airway device 64a are distinctly different from the structural features of the prior art devices 50 (FIG. 2) and 50a (FIG. 3), which facilitates the establishment of a comparatively enhanced airway for the patient 26, when using the airway device 64a in the manner described below.

In another aspect of the invention, in a first single-cuff embodiment of the hook-shaped airway device 64a, an inflatable distal cuff 90, which, in this embodiment of the device, is located at a distal position on the distal end 82 of the tube 79, spaced inboard from the distal opening 77b, as shown in FIG. 5. The distal cuff 90, which is the only cuff in this embodiment, is located on the distal end 82 of the curved section 72 of the tube 79 in such a manner that no portion of the cuff overlaps, or obstructs, the distal opening 77b.

In an additional aspect of the invention, in a second single-cuff embodiment of the hook-shaped airway device 64a, an inflatable proximal cuff 88, which, in this embodiment of the device, is the only cuff forming a part of the device, and is located at its proximal position on an intermediate portion of the straight section 70 of the tube 79 of the device, as shown in FIG. 5. Further, the proximal cuff 88 is located inboard from the proximal opening 77a by a prescribed proximal distance.

In a further aspect of the invention, in a first double-cuff embodiment of the device 64a, the device includes only the distal cuff 90, in its distal position, and the proximal cuff 88, in its proximal position.

In still another aspect of the invention, in a second double-cuff embodiment of the hook-shaped airway device 64a, an intermediate 88a is selectively located on the curved section 72 of the tube 79 between the proximal opening 77a and the distal cuff 90, at an intermediate location on the tube as shown in FIG. 5. In this double-cuff embodiment, the device 64a includes only the distal cuff 90, as described above, and the intermediate cuff 88a, in their respective distal and intermediate positions.

In still another aspect of the invention, in a triple-cuff embodiment of the hook-shaped airway device 64a, the device includes the three inflatable cuffs 90, 88 and 88a assembled in their respective distal, proximal and intermediate positions on the tube 79, as illustrated in FIG. 5.

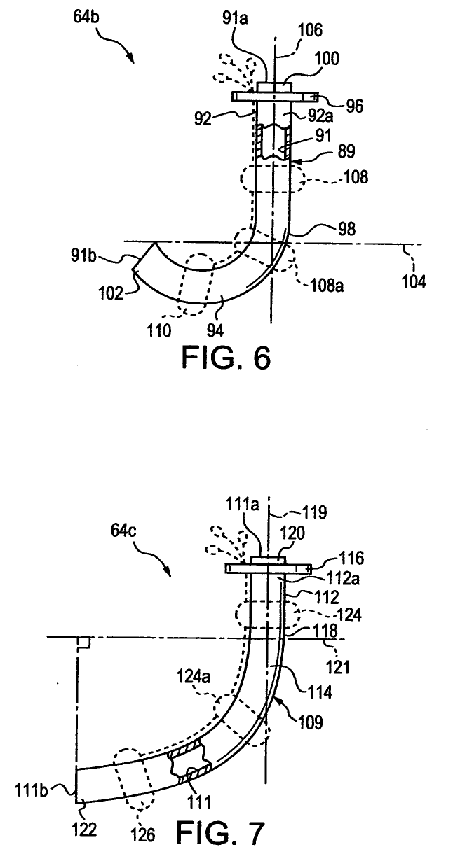

Referring to FIG. 6, in a third embodiment of an inventive airway device, a J-shaped airway device 64b includes a tube 89 formed in a J-shaped configuration. The tube 89 is formed with a hollow passage 91 extending between, and including, a proximal opening 91a of the passage, at a proximal end 92a of the tube, and a distal opening 91b of the passage, at a distal end 102 of the tube. The tube 89 of the airway device 64b is formed with a straight section 92, and a curved section 94. The straight section 92 extends from a flange 96 to a juncture 98 of the straight section with the curved section 94, with an adapter 100 extending outward from the flange.

Without departing from the spirit and scope of the invention, the length of the straight section 92, of the tube 89 of the device 64b, could be consistent with the limited peri-lingular length of the prior art devices 50 (FIG. 2) and 50a (FIG. 3), or with the extended peri-lingular length of the device 64, as described above and as shown in FIG. 4, with or without the flange 96 and the adapter 100.

The curved section 94 of the tube 89 is formed generally in a semi-circular configuration. The curved section 94 is integrally joined with the straight section 92 at the juncture 98 intermediate the proximal end 92a and the distal end 102 of the tube 89. The curved section 94 extends in a generally circular arc away from the juncture 98, with a minor portion of the curved section being located on one side (i.e., to the right) of a centerline 106, or central axis, of the straight section 92. The curved section 94 continues in the generally circular arc, and is located, partially, to an other side (i.e., to the left) of the centerline 106, which is opposite the above-noted one side thereof. It is noted that there are no portions of the curved section 94 of the device 64b which curve in a direction away from the one side (i.e., to the right) of the centerline 106, as the curved section extends from the juncture 98.

The curved section 94 continues in the circular arc and terminates at a distal end 102 thereof, which, in one aspect of the invention, is located a prescribed J-shaped curvilinear distance from the juncture 98. In addition, the distal end 102 of the curved section 94 extends the farthest to the other side (i.e., to the left) of the centerline 106, and is located along a line 104, which is perpendicular to the centerline.

It is noted that, as shown in FIG. 6, the farthest extension of the curved section 94 to the one side (i.e., to the right) of the centerline 106, and the farthest extension of the curved section 94 to the other side (i.e., to the left) of the centerline, are coincidentally located on the line 104. Further, the major curved portion, and nearly all, of the curved section 94 is located on the other side (i.e., to the left) of the centerline 106, and below the line 104.

The above-described structural features of the airway device 64b are distinctly different from the structural features of the prior art devices 50 (FIG. 2) and 50a (FIG. 3), which facilitates the establishment of a comparatively enhanced airway for the patient 26, when using the airway device 64b in the manner described below.

In another aspect of the invention, in a first single-cuff embodiment of the J-shaped airway device 64b, an inflatable distal cuff 110, which, in this embodiment, is located at its distal position on the distal end 102 of the tube 89, spaced inboard from the distal opening 91b, as shown in FIG. 6. The distal cuff 110, which is the only cuff in this embodiment, is located on the curved section 94 of the tube 89 in such a manner that no portion of the cuff overlaps, or obstructs, the distal opening 91b.

In an additional aspect of the invention, in a second single-cuff embodiment of the J-shaped airway device 64b, an inflatable proximal cuff 108, which, in this embodiment of the device, is located at its proximal position on an intermediate portion of the straight section 92 of the tube 89, as shown in FIG. 6. The proximal cuff 108 is located inboard from the proximal opening 91a by a prescribed proximal distance. Further, the proximal cuff 108, at its proximal position, is the only cuff in this embodiment.

In a further aspect of the invention, in a first double-cuff embodiment of the device 64b, the device includes only the distal cuff 110, in its distal position, and the proximal cuff 108, in its proximal position on the tube 89.

In another aspect of the invention, in a second double-cuff embodiment of the J-shaped airway device 64b, an intermediate cuff 108 may be selectively located on the curved section 94 of the tube 89 between the proximal opening 91a and the distal cuff 110, at an intermediate location on the tube as shown in FIG. 6. In this double-cuff embodiment, the device 64b includes only the distal cuff 110, as described above, and the intermediate cuff 108a, each in their respective distal and intermediate positions.

In still another aspect of the invention, in a triple-cuff embodiment of the J-shaped airway device 64b, the device includes the three inflatable cuffs 110, 108 and 108a, assembled in their respective distal, proximal and intermediate positions on the tube 89, as illustrated in FIG. 6.

As shown in FIG. 7, in a fourth embodiment of an inventive airway device, a curved airway device 64c includes a tube 109 which is formed with a hollow passage 111 extending between, and including, a proximal opening 111a of the passage, at a proximal end 112a of the tube, and a distal opening 111b of the passage, at a distal end 122 of the tube. The tube 109 of the airway device 64c is formed with a straight section 112, and a curved section 114. The straight section 112 extends from a flange 116 to a juncture 118 of the straight section with the curved section 114, with an adapter 120 extending outward from the flange.

Without departing from the spirit and scope of the invention, the length of the straight section 112, of the tube 109, could be consistent with the limited peri-lingular length of the prior art devices 50 (FIG. 2) and 50a (FIG. 3), or with the extended peri-lingular length of the device 64, as described above and as shown in FIG. 4, with or without the flange 116 and the adapter 120.

The curved section 114 of the tube 109 is formed in a generally quarter-circular configuration, as indicated in FIG. 7, and is integrally joined with the straight section 112 at the juncture 118. The curved section 114 extends in a generally circular arc away from the juncture 118 intermediate the proximal end 112a and the distal end 82 of the tube 109. A small portion of the curved section 114 is located on one side (i.e., to the right) of a centerline 119, or central axis, of the straight section 112. The curved section 114 continues in the generally circular arc, and is located, partially and more fully, on an other side (i.e., to the left) of the centerline 119, which is opposite the above-noted one side thereof.

It is noted that there are no portions of the curved section 114 of the tube 109 which curve in a direction away from the one side (i.e., to the right) of the centerline 119, as the curved section extends from the juncture 118.

The curved section 114 continues in the circular arc and terminates at a distal end 122 and the distal opening 111b thereof, which, in one aspect of the invention, is located a prescribed curvilinear distance from the juncture 118. In addition, the distal end 122 of the curved section 114 extends the farthest to the other side (i.e., to the left) of the centerline 119.

It is noted that, as shown in FIG. 7, the major curved portion, and nearly all, of the curved section 114 is located on the other side (i.e., to the left) of the centerline 119, and the distal end 122 is located below a horizontal line 121, which is perpendicular with the centerline 119 at the juncture 118. A vertical line 123, which is perpendicular with the horizontal line 121, is coincidental with the farthest extension of the distal end 122 of the curved section 114 to the other side (i.e., to the left) of the centerline 119.

The above-described structural features of the airway device 64c are distinctly different from the structural features of the prior art devices 50 (FIG. 2) and 50a (FIG. 3), which facilitates the establishment of a comparatively enhanced airway for the patient 26, when using the airway device 64c in the manner described below.

In another aspect of the invention, in a first single-cuff embodiment of the curved airway device 64c, an inflatable distal cuff 126, which, in this embodiment of the device, is located at its distal position on the distal end 122 of the tube 109, spaced inboard from the distal opening 111b, as shown in FIG. 7. The distal cuff 126, which is the only cuff in this embodiment, is located on the curved section 114 of the tube 109 in such a manner that no portion of the cuff overlaps, nor obstructs, the distal opening 111b.

In an additional aspect of the invention, in a second single-cuff embodiment of the curved airway device 64c, an inflatable proximal cuff 124, which, in this embodiment, is located at its proximal position on an intermediate portion of the straight section 112 of the tube 109, as shown in FIG. 7. The proximal cuff 124 is located inboard from the proximal opening 111a by a prescribed proximal distance. Further, the proximal cuff 124, at its proximal position, is the only cuff in this embodiment.

In a further aspect of the invention, in a first double-cuff embodiment of the device 64c, the device includes only the distal cuff 126, in its distal position, and the proximal cuff 124, in its proximal position.

In still another aspect of the invention, in a second double-cuff embodiment of the curved airway device 64c, an intermediate cuff 124 is selectively located on the curved section 114 of the tube 109 between the proximal opening 111a and the distal cuff 126, at an intermediate location on the tube as shown in FIG. 7. In this double-cuff embodiment, the device 64c includes only the distal cuff 126, as described above, and the intermediate cuff 124a, each in their respective distal and intermediate positions.

In still another aspect of the invention, in a triple-cuff embodiment of the curved airway device 64c, the device includes the three inflatable cuffs 126, 124 and 124a, assembled in their respective distal, proximal and intermediate positions, as illustrated in FIG. 7.

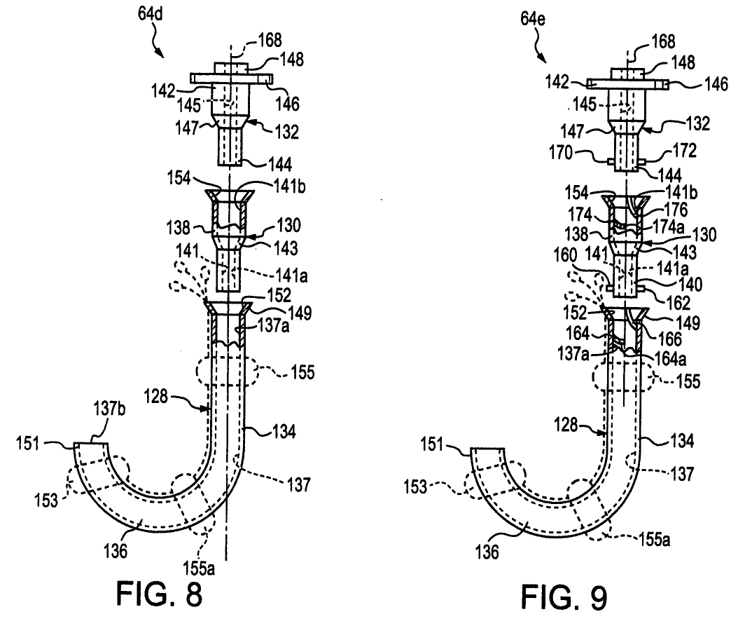

Referring to FIG. 8, a fifth embodiment of the invention shows a hollow adjustable airway device 64d, which provides facility for increasing, or decreasing, a proximal portion of the device, as described hereinbelow. In particular, the airway device 64d, as illustrated, includes a hollow contoured element 128, or tube, which can be used interchangeably with a first hollow axially-straight element 130, or tube, and a second hollow axially-straight element 132, or tube, which can be used interchangeably with the hollow contoured element 128 or with the combination of the hollow contoured element and the first element 130.

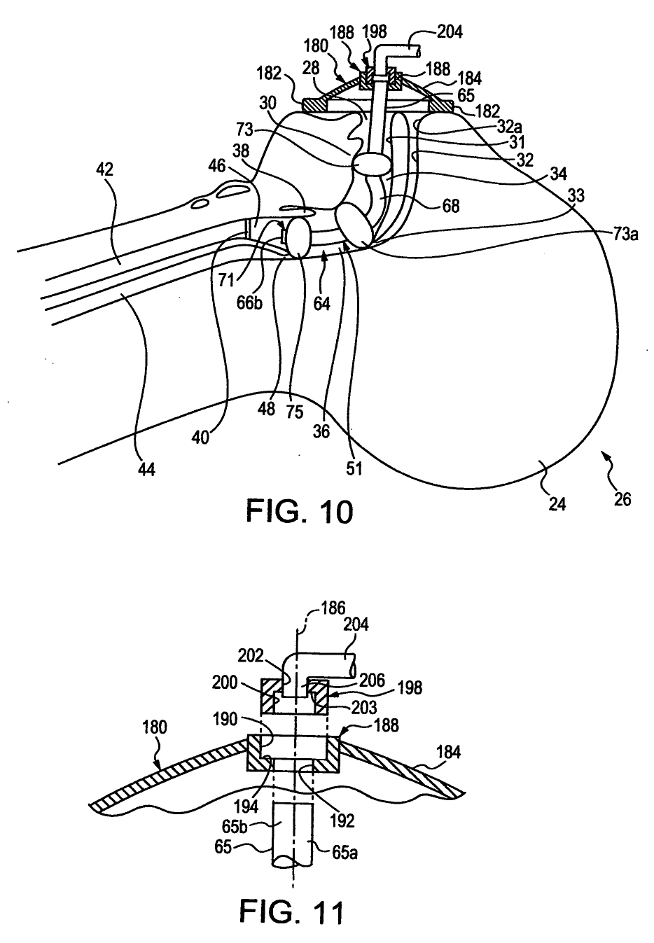

In a first optional use of the airway device 64d, the hollow contoured element 128 is assembled with a distal section of the second hollow element 132 to form a two-piece version of the airway device 64e. In the two-piece version, a proximal section of the second hollow element 132 forms a limited proximal portion of the device 64d, which extends outward from the patient's mouth 28 when the device 64d is in place within the patient's mouth, oral cavity 34 and oropharyngeal area 36, in the same manner as the proximal portion 65a of the airway device 64 (FIG. 10).

Further, in a second optional use of the airway device 64d, the hollow contoured element 128 is in assembly with the distal section of the first hollow element 130, with the proximal section of the first hollow element in assembly with the distal section of the second hollow element 132 to form a three-piece version of the airway device 64e having an extended proximal portion.

In the three-piece version, the proximal sections of the assembled first hollow element 130 and the second hollow element 132 form the above-noted extended proximal portion of the device 64d, which extends outward from the patient's mouth 28 when the three-piece version of the device 64d is in place within the patient's mouth, oral cavity 34 and oropharyngeal area 36, in the same manner as the proximal portion 65a of the airway device 64 (FIG. 10).

Without departing from the spirit and scope of the invention, the contoured element 128 could be shaped in similar fashion to conform to the shape of an airway device, such as, but not limited to, any of the extended airway device 64 (FIG. 4), the hook-shaped airway device 64a (FIG. 5), the J-shaped airway device 64b (FIG. 6), or the airway device 64c (FIG. 7). For purposes of description and illustration herein, the contoured element 128 will be described herein, and illustrated in FIG. 8, as a J-shaped element.

The J-shaped element 128, or hollow contoured element, is formed with a proximal end 129 and a distal end 131, a straight section 134 and a curved section 136, and a hollow passage 137, extending through the element. At least a proximal upper portion 137a of the hollow passage 137 is formed with a prescribed internal diameter.

The first hollow axially-straight element 130 is formed with a body in the shape of a hollow through-sleeve, having a hollow upper, or proximal, section 138 and a hollow lower, or distal, section 140. The element 130 is formed with a hollow passage 141 extending therethrough, which is formed with a lower, or distal, portion 141a, of the hollow passage 141, having a first inner diameter, and an upper, or proximal, section 141b, of the hollow passage, formed at a second inner diameter, which is greater than the first outer diameter and equal to the above-noted prescribed internal diameter.

The second axially-straight element 132 is also formed with a body in the shape of a linear hollow through-sleeve, having a hollow upper, or proximal, section 142 and a hollow lower, or distal, section 144, with a hollow passage 145 extending therethrough. A flange 146 and an adapter 148 are located at the upper end of the second element 132. The inner diameter 145 of the second element 132 can be of any size and variance along its axial length, within the structural limits permitted by the exterior dimensions of the second element. Also, the inner diameter of the second element 132 must be of sufficient dimension to provide an acceptable airway for the patient 26.

The hollow lower section 140 of the first element 130 is formed with an outer diameter, which is slightly smaller than the prescribed internal diameter of a hollow upper portion, or passage, 137a of the hollow passage 137 of the J-shaped element 128, whereby the lower section 140 will fit snugly into, and be held by friction-fit assembly within, the upper portion 137a.

As noted above, the inner diameter of the lower portion 141a of the hollow passage 141 of the first element 130 is smaller than the inner diameter of the upper portion 141b of the hollow passage. In turn, the inner diameter of the upper portion 141b is the same as the prescribed internal diameter of the upper portion 137a of the hollow passage 137 of the J-shaped element 128.

The hollow lower, or distal, section 144 of the second element 132 is formed with an outer diameter, which is the same as the diameter of the lower, or distal, section 140 of the first element 130. Consequently, the outer diameter of the lower section 144 is also slightly smaller than the prescribed internal diameter of the upper portion 137a of the hollow passage 137 of the J-shaped element 128. As noted above, the structure of the second element 132 facilitates the positioning of the lower section 144 of the second element 132 snugly into, to be held by friction-fit assembly within, either the upper portion, or passage, 137a of the hollow passage 137 of the J-shaped element 128, or the upper portion 141b of the hollow passage 141 of the first element 130.

As noted above, and without departing from the spirit and scope of the invention, only the upper portion 137a of the hollow passage 137 of the J-shaped element 128 requires a diameter sufficient to receive, in a friction-fit assembly, the lower, or distal, sections 140 and 144 of the first and second elements 130 and 132, respectively. The inner diameter of the remainder of the hollow passage 137 can be the same as, smaller than, or larger than, the prescribed internal diameter of the upper portion 137a, provided that such diameter is sufficient for ventilation of the patient 26.

As illustrated in FIG. 8, the outer diameter of the upper section 138 of the first element 130 is greater than the outer diameter of the lower section 140 of the first element, with a bevelled transition surface 143 being formed at a juncture between the upper and lower sections 138 and 140. In similar fashion, the outer diameter of the upper section 142 of the second element 132 is greater than the outer diameter of the lower section 144 of the second element, with a bevelled transition surface 147 being formed at a juncture between the upper and lower sections 142 and 144.

With the upper section 138 of the first element 130 being formed with a larger outer diameter, sufficient volume is provided in the upper section of the first element to facilitate the formation of the inner diameter, and depth, of the upper portion 141b of the hollow passage 141 to be the same as the prescribed internal diameter, and depth, of the upper portion 137a of the hollow passage 137 of the J-shaped element 128.

With this construction, the outer diameter of the lower section 144 of the second element 132 can be formed with generally the same diameter as the outer diameter of the lower section 140 of the first element 130, so that, if desired, the second element 132 can be assembled directly with either the J-shaped element 128 or the first element 130.

In either the two-piece version, or the three-piece version, of the airway device 64e, as described above, the flange 146 and the adaptor 148 of the second element 132 will always appear at the upper, or proximal, end of either version of the airway device 64e.

As described above, the upper, or proximal, ends of the J-shaped element 128 and the first element 130 include flared sections which form funnel-shaped surfaces 152 and 154, respectively, which further enhance the frictional-fit of the first element 130 and the second element 132 with the J-shaped element 128 and the first element, respectively.

It is noted that the outer surfaces of the lower sections 140 and 144 of the first element 130 and the second element 132, respectively, could each be tapered inward, or formed with a funnel shape, extending below the respective bevelled surfaces 143 and 147, inward toward the respective distal end of the respective first and second elements, to fit frictionally into correspondingly tapered, or funnel-shaped, inner surfaces formed in place of the upper portions 137a and 141b of the J-shaped element 128 and the first element 130, respectively, without departing from the spirit and scope of the invention.

In another aspect of the invention, in a first single-cuff embodiment of the adjustable airway device 64d, an inflatable distal cuff 153, which, in this embodiment of the device, is located at its distal position on the distal end 151 of the J-shaped element 128, spaced inboard from a distal opening 137b of the J-shaped opening, as shown in FIG. 8. The distal cuff 153, which is the only cuff in this embodiment, is located on the curved section 136 in such a manner that no portion of the cuff overlaps, or obstructs, a distal opening of the J-shaped element 128.

In an additional aspect of the invention, in a second single-cuff embodiment of the curved airway device 64d, an inflatable proximal cuff 155, which, in this embodiment, is located at its proximal position on an intermediate portion of the straight section 134 of the J-shaped element 128, and near the proximal end 149 thereof. The proximal cuff 155 is located inboard from the proximal end 149 by a prescribed proximal distance. Further, the proximal cuff 155, at its proximal position, is the only cuff in this embodiment.

In a further aspect of the invention, in a first double-cuff embodiment of the device 64d, the device includes only the distal cuff 153, in its distal position, and the proximal cuff 155, in its proximal position.

In still another aspect of the invention, in a second double-cuff embodiment of the curved airway device 64d, an intermediate cuff 155 is selectively located on the curved section 136 between the proximal end 149 and the distal cuff 153, at an intermediate location on the J-shaped element 128 as shown in FIG. 8. In this double-cuff embodiment, the device 64d includes only the distal cuff 153, as described above, and the intermediate cuff 155a, each in their respective distal and intermediate positions.

In still another aspect of the invention, in a triple-cuff embodiment of the airway device 64d, the device includes the three inflatable cuffs 153, 155 and 155a, assembled in their respective distal, proximal and intermediate positions on the J-shaped element 128, as illustrated in FIG. 8.

As shown in FIG. 9, a sixth embodiment of the invention, shows a hollow adjustable airway device 64e, which provides facility for increasing, or decreasing, the extended peri-lingular length of the device. The airway devices 64d and 64e are similar and include many of the same structural features and elements. Therefore, only the portions of the device 64e, which differ from the portions of the device 64d, will be described in detail below, with the same numerals being assigned to common elements of the two devices. It to be understood that, except with regard to the additional features of the device 64e as described below, the device 64e functions in the same manner as the device 64d, and such common functions will not be described further hereinbelow.

Regarding the first element 130, a pair of projections 160 and 162 are formed on diametrically opposite sides of the outer surface of the lower section 140 of the first element, and extend radially outward from the diametrically opposite sides.

A pair of spirally-formed grooves 164 and 166 are formed in diametrically opposite sides of the upper portion 137a of the hollow passage 137 of the J-shaped element 128. The upper, or proximal, ends of each of the grooves 164 and 166 are open at the upper, or proximal, end of the J-shaped element 128. The lower, or distal, ends of each of the grooves 164 and 166, as shown, and as represented, by a groove extension 164a of the groove 164 in FIG. 9, extend partially circumferentially around the inner wall of the hollow upper portion 137a of the J-shaped element 128, in a plane which is perpendicular to an axis 168 of the straight section 134 of the J-shaped element 128.

As the lower section 140 of the first element 130 is moved axially into the hollow upper portion 137a of the J-shaped element 128, the pair of projections 160 and 162 are aligned with the open proximal ends of the respective grooves 164 and 166 of the J-shaped element 128, whereafter the projections can be moved into the respective grooves.

The first element 130 is rotated about the axis 168 as the lower section 140 is moved into the hollow upper portion 137a of the J-shaped element 128, whereby the projections 160 and 162 follow the path defined by the spiral of the grooves 164 and 166.

Upon continued rotation of the first element 130, the projections 160 and 162 reach the distal end of the grooves 164 and 166, respectively, whereby the hollow lower section 140 can not be moved any farther axially into the hollow upper portion 137a of the J-shaped element 128. Upon continued rotation of the first element 130, the projections 160 and 162 are moved circumferentially about the axis 168 to distal terminations of the groove extensions, such as the groove extension 164a. In this manner, the first element 130 is assembled, and secured, with the hollow upper portion 137a of the J-shaped element 128, and precluded from axial movement relative thereto.

Either of the groove extensions, such as the groove extension 164a, in conjunction with the respective projection 160 or 162, provide a means for precluding movement by the first element 130, including the first-element projection 160 or 162, axially from assembly with the hollow upper portion, or passage, 137a of the J-shaped element 128.

Regarding the second element 132, a pair of projections 170 and 172 are formed on diametrically opposite sides of the outer surface of the lower section 144 of the second element, and extend radially outward from diametrically opposite sides thereof.

A pair of spirally-formed grooves 174 and 176 are formed in diametrically opposite sides of the upper portion 141a of the hollow passage 141 of the first element 130. The upper, or proximal, ends of each of the grooves 174 and 176 are open at the upper, or proximal, end of the first element 130. The lower, or distal, ends of each of the grooves 174 and 176, as shown, and as represented, by a groove extension 174a of the groove 174 in FIG. 9, extend partially circumferentially around the inner wall of the hollow upper portion 141a of the first element 130, in a plane which is perpendicular to the axis 168.

As the lower section 144 of the second element 132 is moved into the hollow upper portion 141b of the first element 130, the pair of projections 170 and 172 are aligned with the open proximal ends of the respective grooves 174 and 176 of the first element 130, whereby the projections can be moved into the respective grooves.

The second element 132 is rotated about the axis 168 as the lower section 144 is moved into the hollow upper portion 141b of the first element 130, whereby the projections 170 and 172 follow the path defined by the spiral of the grooves 174 and 176, respectively, to the base thereof. Eventually, the projections 170 and 172 reach, and are moved into, the respective groove extensions, such as the groove extension 174a, of the grooves 174 and 176, respectively.

Upon continued rotation of the second element 132, the hollow lower section 144 does not move any farther axially into the upper portion 141a of the first element 130, but the projections 170 and 172 are moved circumferentially about the axis 168 to distal terminations of, and within, the groove extensions, such as the groove extension 174a. In this manner, the second element 132 is assembled, and secured, with the first element 130, and precluded from axial movement relative thereto.

Either of the groove extensions, such as the groove extension 174a, in conjunction with the respective projection 170 or 172, provide a means for precluding movement by the second element 132, including the second-element projection 170 or 172, axially from assembly with the upper portion, or passage, 141b of the first element 130.

As noted above, and as shown in FIGS. 8 and 9, the upper, or proximal, ends of the J-shaped element 128 and the first element 130 include flared sections, which form the funnel-shaped surfaces 152 and 154, respectively. Also, the first and second elements 130 and 132, respectively, are formed with the bevelled surfaces 143 and 147, respectively.

It is noted that, without departing from the spirit and scope of the invention, the upper, or proximal, ends of the J-shaped element 128 and the first element 130 could each be formed without the flared sections, and the first element and the second element 132 could be formed without the bevelled surfaces 143 and 147, respectively. In this instance, the flared sections of the J-shaped element 128 and the first element 130 are replaced with respective non-flared upper, or proximal, surfaces located in respective planes, each of which are perpendicular to the axis 168.

Also, in this instance, the bevelled surfaces 143 and 147 of the first and second elements 130 and 132, respectively, are replaced with respective under surfaces, located in respective planes, each of which are perpendicular to the axis 168. With this structure, when the first element 130 is assembled with the J-shaped element 128, an outer, under surface of the upper portion 138 of the first element will engage and abut an upper, or proximal, surface of the J-shaped element, and an outer, under surface of the upper section 142 of the second element 132 will engage an upper, or proximal, surface of the first element.

As shown in FIG. 10, the hollow extended airway device 64 (FIG. 4) is inserted through the patient's mouth 28, oral cavity 34, and oropharyngeal area 36, such that the distal end 71 of the tube 51 is located in an area directly adjacent the vocal cords 40 and the glottic opening 46.

In one aspect of the invention, when the airway device 64 is positioned as shown in FIG. 10, the distal opening 66b of the tube 51 is located well beyond the epiglottis 38, including a free-end tip thereof, toward and immediately adjacent the glottic opening 46 and the vocal cords 40. This structure facilitates the flow of air, through the hollow passage 66, past, and without obstruction from, the epiglottis 38.

The ability to locate the distal end 71 of the tube 51 in this position, as provided by the above-described extended length of the curved section 68, facilitates the placement of an unobstructed airway, within, and through, the patient's oral cavity 34 and oropharyngeal area 36, and between the patient's mouth 28 and the area including the glottic opening 46 and the vocal cords 40. This placement of the airway device 64 allows uninterrupted air flow past the epiglottis 38, and into the patient's trachea 42, between a location external of the patient 26 and the patient's lungs (not shown), thereby facilitating successful and unobstructed ventilation of the patient 26.

In another aspect of the invention, independently of the above-described curved section 68 with the extended distal end 71, the straight section 65 of the tube 51 is formed with the extended peri-lingular length. With this structure, the proximal portion 65a of the straight section 65 extends outward from the mouth 28 of the patient 26, following placement of the curved section 68, and a portion of the straight section through the patient's mouth, in the illustrated position, shown in FIG. 10. The extended peri-lingular length of the straight section 65 of the tube 51 provides facility for a care giver to access at least the proximal portion 65a of the tube, externally of the patient 26, for utilization by the care giver in preparing the patient 26 for, and during the process of, ventilating the patient.

In one example of such utilization of the straight section 65 with the extended peri-lingular length, the mouth 28 of the patient 26 remains open to facilitate concomitant oral suctioning, if such is required during ventilation of the patient.

Another example of utilization of the extended peri-lingular length of the straight section 65 is illustrated in FIGS. 10 and 11, where a face mask 180 is placed, in a sealing arrangement, about the patient's mouth 28 and outer nasal cavity opening 32a to preclude the inward flow or backflow of air through the patient's mouth and nasal cavity 32, other than through the hollow passage 66 of the tube 51. It is noted that the face mask 180 could be placed over the patient's mouth 28 only, i.e., exclusive of the outer nasal cavity opening 32a, without departing from the spirit and scope of the invention.

The face mask 180 is formed of a generally compliant material, and is further formed with a continuous bead 182 (FIG. 10) around a base of the mask. An enclosure wall 184 of the mask 180 is formed integrally with the bead 182, and extends outward from a common side of the bead, and centrally toward, and about, an axis 186 of the mask.

A receptor 188 is formed integrally with the wall 184, and is located about the axis 186. The receptor 188 is formed with a cup-like opening 190, having a prescribed shape and prescribed dimensions, with the opening facing away from the mask 180. The prescribed shape of the receptor 188 could be, for example, circular, square, rectangular, or other shapes without departing from the spirit and scope of the invention. The receptor 188 is further formed with a through-passage 192, which is centrally located through a floor 194 of the receptor, and about the axis 186. The through-passage 192 is formed in a shape and dimensions for sealing receipt of the proximal portion 65a of the straight section 65 of the tube 51, shown in FIGS. 10 and 11, in an embodiment which does not include the flange 67 (FIG. 4) and the adapter 69 (FIG. 4).

A compliant adapter 198 is formed with an inverted cup-like opening 200, and a through-passage 202 in a floor 203 of the adapter, with the exterior of side walls of the adapter being formed in the prescribed shape, and with dimensions which are slightly less than the prescribed dimensions, of the receptor 188.

When the mask 180 is to be used in conjunction with the ventilation of the patient 26, the mask is manipulated so that the proximal portion 65a of the straight section 65 is axially aligned with, and above, the through-passage 192 of the receptor 188. The mask 180 is then moved to insert the proximal portion 65a of the straight section 65 through the through-passage 192, and partially into the cup-shaped opening 190, in an air-tight assembly, as shown in FIG. 10.

A feed tube 204, having a proximal end (not shown) connected, for example, to an ambu bag (not shown), is manipulated to align an angled distal end 206 of the tube with, and to insert the tube within and through, the through-passage 202 of the adapter 198, in an air-tight assembly. The adapter 198 is then inserted into the cup-like opening 190 of the receptor 188 to a position as shown in FIG. 10, whereby the proximal portion 65a of the straight section 65 is axially aligned with, but spaced from, the distal end 206 of the tube 204, within a sealed chamber formed by an air-tight assembly of the adapter with the receptor. In this manner, the ambu bag is coupled to the patient 26 for purposes of ventilating the patient.

Without departing from the spirit and scope of the invention, the airway device 64 can provide, in the alternative, at least three inventive combinations. A first inventive combination includes the curved section 68 of the tube 51 with the extended distal end 71, and with the straight section 65 formed with the limited peri-lingular length (i.e., without the proximal portion 65a). A second inventive combination includes the straight section 65 having the extended peri-lingular length, with the proximal portion 65a, and the curved section 68 formed with the distal end 63 (FIG. 3) (i.e., without the extended distal end 71). A third inventive combination includes the extended distal end 71 of the curved section 68 and the straight section 65, with the extended peri-lingular length, formed by the proximal portion 65a.

In another aspect of the invention, in the above-described first single-cuff embodiment of the device 64, the tube 51 includes the distal cuff 75, as shown in FIG. 10, which is moved through the oral cavity 34 and the oropharyngeal area 36 of the patient 26. During the period when the device 64 is being placed within the oral cavity 34 and the oropharyngeal area 36, the distal cuff 75 is in the deflated state.

When the device 64 is fully placed in the illustrated position of FIG. 10, the distal opening 66a, at the distal end 71, of the tube 51 is located at the glottic opening 46 and the vocal cords 40, as noted above. With this placement, the deflated distal cuff 75 is located about the curved section 68 of the device 64, at the distal end 71 thereof, with no portion of the distal cuff extending over, or obstructing, the distal opening 66b, and with the distal cuff spaced slightly inboard from the distal opening 66b.

As illustrated in FIG. 10, the distal cuff 75 is positioned on the distal end 71 of the curved section 68, slightly inboard, by a prescribed distal distance, from the distal opening 66b, and shows the closeness of the distal opening to the glottic opening 46 and the vocal cords 40. Also, the distal cuff 75 is shown at the glottic opening 46, which means that the distal opening 66b is not in the glottic opening, and not into the trachea 42. It is noted that the distal cuff 75 could be positioned at the extreme distal portion of the distal end 71, which surrounds the distal opening 66a, without departing from the spirit and scope of the invention, provided that no portion of the cuff, deflated or inflated, is obstructing any portion of the distal opening.

After the device 64 has been placed in the position illustrated in FIG. 10, the distal cuff 75 is inflated in a conventional manner. The inflated cuff 75 forms a seal over the esophageal opening to preclude communication between the esophagus 44 and the oropharyngeal area 36 when the distal end 71 is located at the glottic opening. The seal prevents air, which, during ventilation of the patient 26, is moving through the distal opening 66b of the tube 51, from undesirably entering the patient's esophagus 44 and stomach (not shown).

In addition, as shown in FIG. 10, the inflated distal cuff 75 displaces and holds the patient's epiglottis 38 in a non-obstructing location spaced from the tube 51, when the distal end 71 of the tube is located at the glottic opening 46. Further, as the cuff 75 is being inflated, the inflating cuff provides a centralizing adjustment, if necessary, of the distal end 71 of the tube 51 to locate the distal opening 66b of the tube in general alignment with the patient's glottic opening 46 and the vocal cords 40, when the distal end is at the glottic opening. Still further, the inflated cuff 75 provides surrounding and strengthening support for distal end 71 of the tube 51, when the distal end is at the glottic opening 46.

Also, the inflated distal cuff 75 forms a seal around the exterior of the tube 51 and over the glottic opening 46 and the vocal cords 40 to preclude communication between the oropharyngeal area 36 and the glottic opening and the vocal cords, when the distal end 71 of the tube is located at the glottic opening and the vocal cords 40. Formation of this seal precludes any backflow of air, which is appearing at the distal opening 66b of the tube 51, from undesirably flowing through the oropharyngeal area 36, the oral cavity 34, and eventually out of the patient's mouth 28. The sealing of the glottic opening 46 and the vocal cords 40, in this manner, insures that all of the air flowing through the device 64 will flow through the patient's trachea 42 and into and out of the lungs, thereby enhancing the ventilating of the patient 26.

In another aspect of the invention, in the above-described second single-cuff embodiment of the device 64, the tube 51 includes the deflated proximal cuff 73 in its proximal position, inboard from the proximal opening of the tube by the prescribed proximal distance, with the distal end 71 of the tube located at the glottic opening 46 and the vocal cords 40, as shown in FIG. 10. After the device 64 has been positioned as shown in FIG. 10, the proximal cuff 73, which is now located in the oral cavity 34, is inflated in a conventional manner, whereby the cuff inflates between an intermediate top portion of the patient's tongue 30 and palate 31. The inflated cuff 73 forms a seal in the oral cavity 34 to preclude communication between the oropharyngeal area 36 and the patient's mouth 28. In this manner, the inflated proximal cuff 73 prevents backflow of air through the patient's mouth 28, thereby providing enhanced ventilation of the patient 26.

In still another aspect of the invention, in the above-described first double-cuff embodiment of the device 64, the proximal cuff 73 is located in the above-noted proximal position, and in a deflated state, and the deflated distal cuff 75 is located at its above-noted distal position on the distal end 71 of the tube 51.

After the device 64 has been positioned as shown in FIG. 10, the distal cuff 75 is inflated, and is in a sealing position about the esophageal opening 48, the glottic opening 46 and the vocal cords 40, forming seals in the manner described above with respect to the first single-cuff embodiment of the device.

Also, in the first double-cuff embodiment of the device 64, the proximal cuff 73 is inflated in a conventional manner, whereby the cuff inflates between an intermediate top portion of the patient's tongue 30 and palate 31 to form the seal in the oral cavity 34 as described above, thereby preventing backflow of air through the patient's mouth 28. In this manner, in the first double-cuff embodiment of the device 64, provides enhanced ventilation of the patient 26.

In another aspect of the invention, in the above-described second double-cuff embodiment of the device 64, the device includes the distal cuff 75, as shown in FIG. 10, and the intermediate cuff 73a in its intermediate position, which is located in the oropharyngeal area 36 of the patient 26. During the period when the device 64 is being placed within the oral cavity 34 and the oropharyngeal area 36, the distal cuff 75 and the intermediate cuff 73a are in the deflated state.

After the device 64 has been placed in the position illustrated in FIG. 10, the distal cuff 75 is inflated, and forms the seals about the esophageal opening 48, the glottic opening 46 and the vocal cords 40, in the manner described above with respect to the first single-cuff embodiment of the device.

Also, the intermediate cuff 73a is inflated in a conventional manner, whereby the cuff inflates between the back of the patient's tongue 30 and a wall adjacent the inner nasal-cavity opening 33 to form a seal the patient's oropharyngeal area 36 and oral cavity 34 to preclude communication between the oropharyngeal area and the oral cavity when the distal end 71 is located at the glottic opening 46 and the vocal cords 40 mouth 28 and nasal cavity 32. In this manner, the inflated intermediate cuff 73a prevents the backflow of air from the oropharyngeal area 36, and eventually through the patient's mouth 28, thereby providing enhanced ventilation of the patient 26.

Also, when the intermediate cuff 73a is inflated in a conventional manner, as noted above, the intermediate cuff inflates between the back of the patient's tongue 30 and a wall adjacent the inner nasal-cavity opening 33 to form a seal over the inner nasal-passage opening to preclude communication between the oropharyngeal area 36 and the inner nasal-cavity opening when the distal end 71 is located at the glottic opening 46 and the vocal cords 40. In this manner, the inflated intermediate cuff 73a prevents the backflow of air from the oropharyngeal area 36, and eventually through the patient's nasal passage 32, thereby providing enhanced ventilation of the patient 26.

In still another aspect of the invention, in the triple-cuff embodiment of the curved airway device 64c, the device includes the three inflatable cuffs 75, 73 and 73a, each of which is assembled in the respective distal, proximal and intermediate positions, as illustrated in FIG. 10, and as described above. Each of the cuffs 75, 73 and 73a function in the same manner as described above regarding the first single-cuff embodiment, the second single-cuff embodiment and the second double-cuff embodiment, respectively, of the device 64.

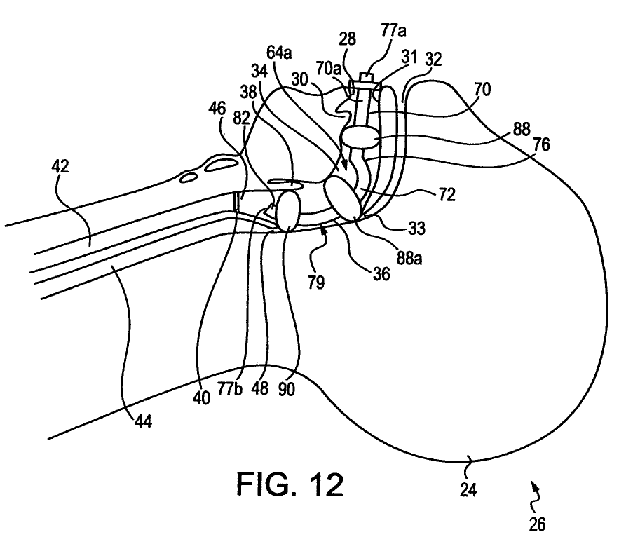

As shown in FIG. 12, the hook-shaped airway device 64a is located within the patient's oral cavity 34 and oropharyngeal area 36, and the straight section 70 of the tube 79 is formed with the limited peri-lingular length. The straight section 70 could be formed with the extended peri-lingular length, in the manner described above with respect to the extended peri-lingular length of the device 64 (FIG. 4). The extended peri-lingular length of the device 64a, independent of any other structural features of the device, provides the advantages and benefits described above with respect to the extended peri-lingular length of the device 64.

Independent of any other structural features of the device 64a, the contour of the hook shape of the tube 79, the curvilinear distance of the distal end 82 from the juncture 76, and the linear length of the straight section, facilitates the placement of the distal end at the patient's glottic opening 46 and vocal cords 40. This placement of the device 64a provides an unobstructed airway at least between the patient's mouth 28 and the patient's glottic opening 46 and vocal cords 40, for an enhanced ventilation of the patient 26 in the same manner as that described above with respect to the airway device 64 (FIG. 4).

In addition, the device 64a could include the extended peri-lingular length of the straight section 70, with or without the extended distal end 82, without departing from the spirit and scope of the invention.

As further shown in FIG. 12, the distal cuff 90, the proximal cuff 88 in its proximal position, and the intermediate cuff 88a in its intermediate position are positioned on the device 64a in locations similar to the locations of the cuffs 75, 73 and 73a, respectively, on the device 64 (FIG. 4).

In the device 64 (FIG. 4), the cuffs 75, 73 and 73a are used in various cuff embodiments, in the manner described above, with respect to FIG. 10. In the device 64a, the distal cuff 90 is used in a first single-cuff embodiment, the proximal cuff 88 in the proximal position is used in a second single-cuff embodiment, the distal cuff 90 and the proximal cuff 88 in the proximal position are used in a first double-cuff embodiment, the distal cuff 90 and the intermediate cuff 88a in the intermediate position are used in a second double-cuff embodiment, and the three cuffs 90, 88, and 88a are used in a triple-cuff embodiment, all in the manner described above with respect to the use of the cuffs 75, 73 and 73a in conjunction with the device 64.

Thus, the airway device 64a possesses all of the different aspects of the invention as described in detail above with respect to the airway device 64 (FIG. 4).

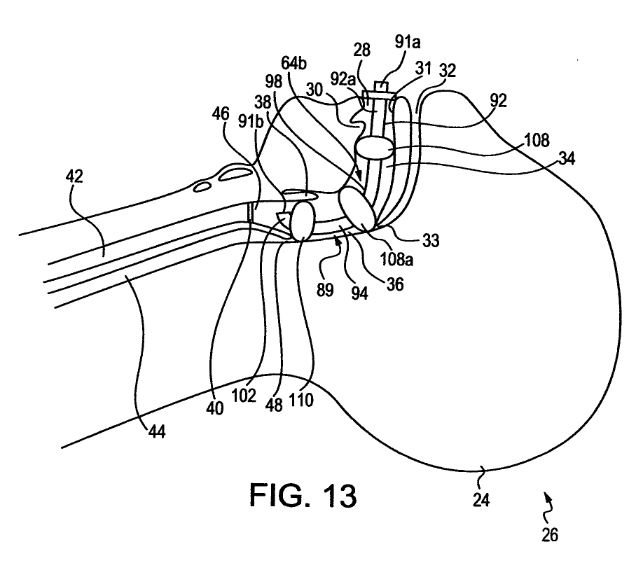

As shown in FIG. 13, the J-shaped airway device 64b is located within the patient's oral cavity 34 and oropharyngeal area 36, and the straight section 92 of the tube 89 is formed with the limited peri-lingular length. The straight section 92 could be formed with the extended peri-lingular length, in the manner described above with respect to the extended peri-lingular length of the device 64 (FIG. 4). The extended peri-lingular length of the device 64b, independent of any other structural features of the device, provides the advantages and benefits described above with respect to the extended peri-lingular length of the device 64.

Independent of any other structural features of the device 64b, the contour of the “J” shape of the straight section 92 and the curved section 94, the curvilinear distance of the distal end 102 from the juncture 98, and the linear length of the straight section, facilitates the placement of the distal end at the patient's glottic opening 46 and vocal cords 40. This placement of the device 64b provides an unobstructed airway at least between the patient's mouth 28 and the patient's glottic opening 46 and vocal cords 40, for an enhanced ventilation of the patient in the same manner as that described above with respect to the airway device 64 (FIG. 4).

In addition, the device 64b could include the extended peri-lingular length of the straight section 92, with or without the distal end 102, without departing from the spirit and scope of the invention.

As further shown in FIG. 13, the distal cuff 110 in its distal position, the proximal cuff 108 in its proximal position, and the intermediate cuff 108a in its intermediate position are positioned on the tube 89 in locations similar to the locations of the cuffs 75, 73 and 73a, respectively, on the device 64 (FIG. 4).

In the device 64 (FIG. 4), the cuffs 75, 73 and 73a are used in various cuff embodiments, in the manner described above, with respect to FIG. 10. In the device 64b, the distal cuff 110 in its distal position is used in a first single-cuff embodiment, the proximal cuff 108 in its proximal position is used in a second single-cuff embodiment, the distal cuff 110 in its distal position and the proximal cuff 108 in its proximal position are used in a first double-cuff embodiment, the distal cuff 110 in its distal position and the intermediate cuff 108a in its intermediate position are used in a second double-cuff embodiment, and the three cuffs 110, 108, and 108a are used in a triple-cuff embodiment, all in the manner described above with respect to the use of the cuffs 75, 73 and 73a, in conjunction with the device 64.

Thus, the airway device 64b possesses all of the different aspects of the invention as described in detail above with respect to the airway device 64 (FIG. 4).

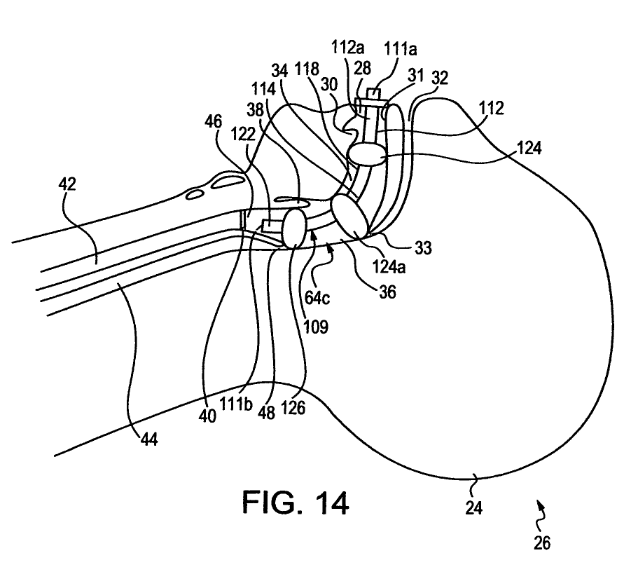

As shown in FIG. 14, the curved airway device 64c is located within the patient's oral cavity 34 and oropharyngeal area 36, and the straight section 112 of the tube 109 is formed with the limited peri-lingular length. The straight section 112 could be formed with the extended peri-lingular length, in the manner described above with respect to the extended peri-lingular length of the device 64 (FIG. 4). The extended peri-lingular length of the device 64c, independent of any other structural features of the device, provides the advantages and benefits described above with respect to the extended peri-lingular length of the device 64.

Independent of any other structural features of the device 64c, the contour of the tube 109, the curvilinear distance of the distal end 122 from the juncture 118, and the linear length of the straight section 112, facilitates the placement of the distal end at the patient's glottic opening 46 and vocal cords 40. This placement of the device 64c provides an unobstructed airway at least between the patient's mouth 28 and the patient's glottic opening 46 and vocal cords 40, for an enhanced ventilation of the patient 26 in the same manner as that described above with respect to the distal end 71 of the airway device 64 (FIG. 4).

In addition, the device 64c could include the extended peri-lingular length of the straight section 112, with or without the distal end 122, without departing from the spirit and scope of the invention.

As further shown in FIG. 14, the distal cuff 126, the proximal cuff 124 in the proximal position, and the intermediate cuff 124a in its intermediate position are positioned on the device 64c in locations similar to the locations of the cuffs 75, 73 and 73a, respectively, on the device 64 (FIG. 4).

In the device 64 (FIG. 4), the cuffs 75, 73 and 73a are used in various cuff embodiments, in the manner described above, with respect to FIG. 10. In the device 64c, the distal cuff 126 in its distal position is used in a first single-cuff embodiment, the proximal cuff 124 in the proximal position is used in a second single-cuff embodiment, the distal cuff 126 and the proximal cuff 124 in the proximal position are used in a first double-cuff embodiment, the distal cuff 126 and the intermediate cuff 124a in its intermediate position are used in a second double-cuff embodiment, and the three cuffs 126, 124, and 124a are used in a triple-cuff embodiment, all in the manner described above with respect to the different cuff embodiments of the cuffs 75, 73 and 73a, in conjunction with device 64.

Thus, the airway device 64c possesses all of the different aspects of the invention as described in detail above with respect to the airway device 64 (FIG. 4).

The six embodiments of the above-described airway devices 64, 64a, 64b, 64c, 64d and 64e each include a respective tube having a straight section, formed about a central axis, a curved section and distal end. For example, the J-shaped airway device 64b (FIG. 6) includes the straight section 92 formed about the central axis 106, and the curved section 94 with the distal end 102.

Each of the airway devices 64 through 64e (i.e., 64, 64a, 64b, 64c, 64d and 64e) include the tube formed with a prescribed length, which extends from the proximal end of the tube to the distal end of the tube, where the prescribed length facilitates the locating of the distal end at the patient's glottic opening 46 and vocal cords 40 when the tube is positioned in the oral cavity and the oropharyngeal area. In addition, each of the airway devices 64 through 64e include the tube formed with a proximal portion at the proximal end of the tube, where the proximal portion extends outward from the patient's mouth 28 a prescribed distance, when the tube is positioned in the oral cavity 34 and the oropharyngeal area 36 of the patient 26 and the distal end of the tube is located at the glottic opening 46 and the vocal cords 40.

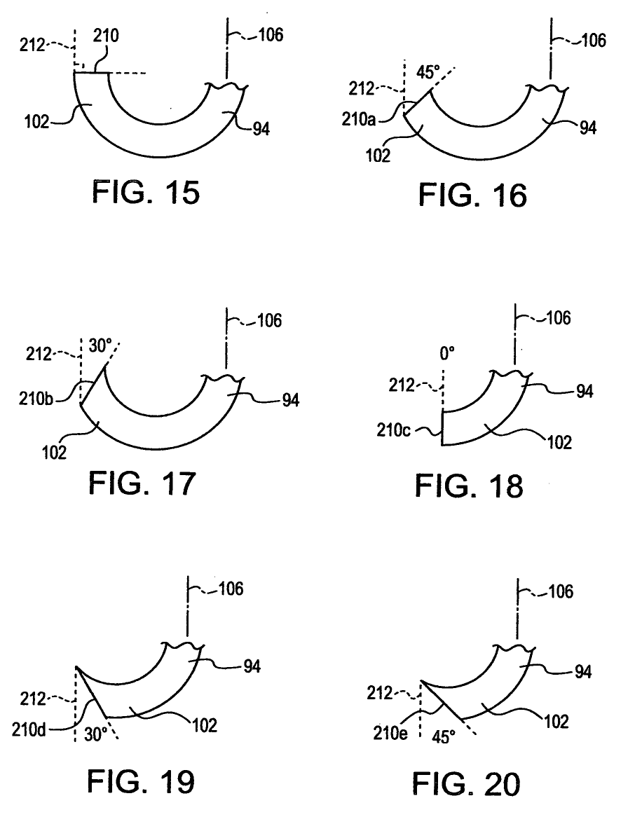

With respect to each of FIGS. 15 through 20, only the distal end of the curved section of the respective airway devices are shown, with the central axis of the respective straight section identified by the number 106. The illustrated portion of the distal end of the curved section will be identified by the number 94, and the distal end will be identified by the number 102, with a free-end termination of the distal end, referred to below as a distal surface, being identified by a series of six respective identifiers 210 through 210e. It is to be understood that the description below, relative to FIGS. 15 through 20, and referring to the configurations of the distal surfaces 210 through 210e, applies to the respective distal surface of each of the six above-described airway devices 64, 64a, 64b, 64c, 64d and 64e.

Referring to FIG. 15, the distal end 102 is formed with a distal surface 210, at an angle of ninety degrees from a plane 212, which is parallel to, and spaced from, the central axis 106. With this configuration, the distal surface 210 is placed at the patient's glottic opening 46 and vocal cords 40, as described above, to provide enhanced ventilation of the patient 26.

As shown in FIG. 16, the distal end 102 is formed with a bevelled surface, which is referred to as an anterior distal surface 210a, at an angle of forty-five degrees from the plane 212, which is parallel to, and spaced from, the central axis 106.

Referring to FIG. 17, the distal end 102 is formed with a bevelled surface, which is referred to as an anterior distal surface 210b, at an angle of thirty degrees from the plane 212, which is parallel to, and spaced from, the central axis 106.

Each of the anterior distal surfaces 210a and 210b are configured, as a bevel, to engage, and enhance the displacement of, the epiglottis 38 when the distal end 102 is being moved through the oropharyngeal area 36, during placement of the respective device 64 through 64e, on which the distal surface is formed. Also, the anterior angle of each of the distal surfaces 210a and 210b provides enhanced ventilation of the patient 26.

As shown in FIG. 18, the distal end 102 is formed with a bevelled surface, which is referred to as a distal surface 210c, at an angle of zero degrees from the plane 212, which is parallel to, and spaced from, the central axis 106. With this configuration, the distal end 102 is placed at the patient's glottic opening 46 and vocal cords 40, as described above, to provide enhanced ventilation of the patient 26.

Referring to FIG. 19, the distal end 102 is formed with a bevelled surface, which is referred to as a posterior distal surface 210d, at an angle of thirty degrees from the plane 212, which is parallel to, and spaced from, the central axis 106.

As shown in FIG. 20, the distal end 102 is formed with a bevelled surface, which is referred to as a posterior distal surface 210e, at an angle of forty-five degrees from the plane 212, which is parallel to, and spaced from, the central axis 106.

Each of the posterior distal surfaces 210d and 210e are configured to engage, and enhance the displacement of, the epiglottis 38 when the distal end 102 is being moved through the oropharyngeal area 36, during placement of its respective device 64 through 64e, on which the distal surface is formed. Also, the posterior angle of the distal surfaces 210d and 210e provide enhanced ventilation of the patient 26.

It is noted that, the angular orientation of the distal surfaces 210a, 210b, 210d and 210e are examples of such orientation of the bevel of the distal surfaces, which are located within an angular range of one hundred and thirty-five degrees, from the angular orientation of the distal surface 210 (FIG. 15) to the angular orientation of the distal surface 210e (FIG. 20). The angular orientation of the bevel of the distal surfaces of any of the devices 64 through 64e could lie within the angular range of the above-noted one hundred and thirty-five degrees without departing from the spirit and scope of the invention.

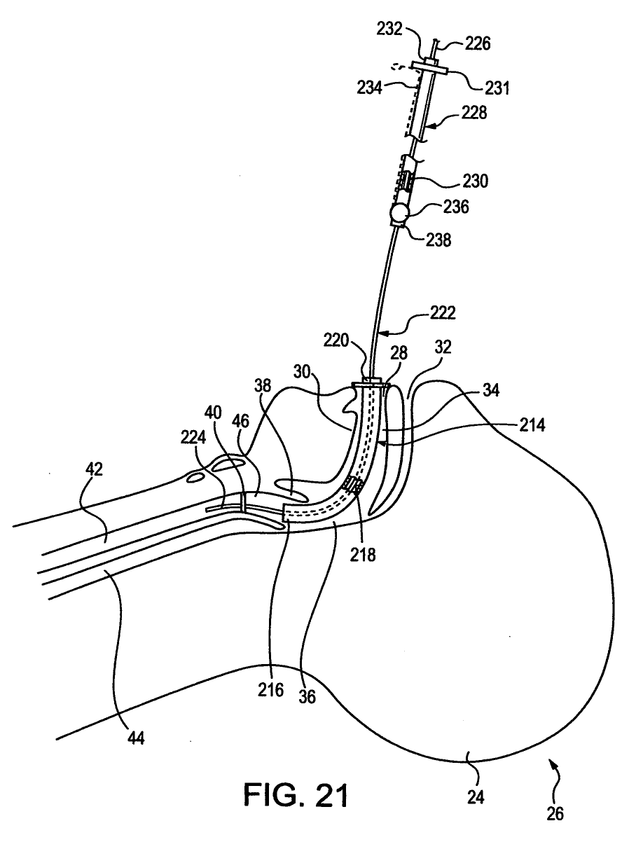

Referring to FIG. 21, an airway device 214 is formed with an extended distal end 216 in the same manner as the above-described devices 64 through 64e. The device 214 is located within the patient's oral cavity 34 and oropharyngeal area 36 in the same manner as the above-described airway devices 64 through 64e, with the distal end 216 of the device 214 being located at the patient's glottic opening 46 and vocal cords 40. The airway device 214 is formed with a hollow passage 218, which is open at a proximal end 220 and the distal end 216 thereof.

After the device 214 has been placed in the position illustrated in FIG. 21, the device is used to provide a guide for a flexible stylette 222 which is guidingly passed through the hollow passage 218 of the device 214. A distal end 224 of the stylette 222 extends outward from the distal end 216 of the device 214 and is located in the patient's trachea 42. A proximal end 226 of the stylette 222 extends outward from the proximal end 220 of the device 214, and from the patient's mouth 28.

Thereafter, the device 214 is withdrawn, leaving an intermediate portion of the stylette 222 in the patient's oral cavity 34 and oropharyngeal area 36, with the distal end 224 of the stylette located in the patient's trachea 42, and the proximal end 226 of the stylette extending outward from the patient's mouth 28.

A hollow endotracheal tube 228 is formed about an axis thereof with a through passage 230, and with a flange 231 and an adapter 232 at a proximal end 234 of the tube. An inflatable cuff 236 is located about the tube 228 near a distal end 238 thereof.

After the device 214 has been withdrawn, the tube 228 is manipulated to place the proximal end 226 of the stylette 222 through the through passage 230, as shown in FIG. 21. The tube 228 is then guidingly moved over the stylette 222 toward, and into, the patient's mouth 28, over the intermediate portion of the stylette within the patient's oral cavity 34 and oropharyngeal area 36. During this movement of the tube 228, the distal end 238 and the cuff 236 are guidingly moved over the stylette and through the patient's glottic opening 46 and vocal cords 40, and into the patient's trachea 42. Thereafter, the stylette 222 may be withdrawn through the patient's mouth 28, leaving the tube 228 in the assembled position, with the inflatable cuff 236 and the distal end 238 of the tube located within the patient's trachea 42, and the flange 230 at the patient's mouth 28. The cuff 236 is then inflated to seal the area within the trachea 42, adjacent the distal end 238 of the tube 228, to prevent undesirable backflow of air through the patient's mouth 28 during the ventilation process.

In an alternative use of the device 214, without departing from the spirit and scope of the invention, the device is positioned in the patient's oral cavity 34 and oropharyngeal area 36, as illustrated in FIG. 21. The endotracheal tube 228 is then inserted through the hollow passage 218 of the device 214, with the distal end 238, and the cuff 236, of the tube positioned in the trachea 42 as noted above. Thereafter the cuff 236 is inflated for the purpose noted above. The device 214 can remain in place during the ventilation process, or it can be removed before beginning the process, leaving the endotracheal tube 228 in the assembled cuff-inflated position in preparation for the ventilation process.

In this manner, any of the devices 64 through 64e can be used in place of the device 214 to facilitate the proper assembly of the tube 228 within the patient 26, without departing from the spirit and scope of the invention. While use of this technique is beneficial for any care giver, regardless of their location, it is particularly useful for care givers who are not in a hospital environment.

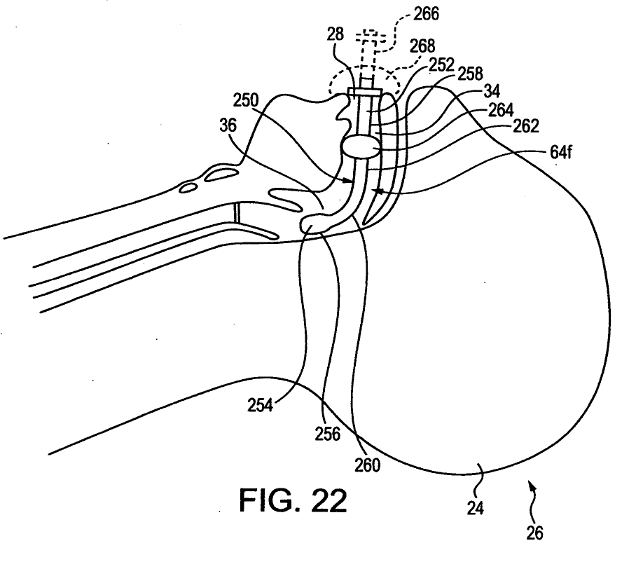

Referring to FIG. 22, a hollow airway device 64f is formed by having a tube 250 extending from a proximal end 252 thereof to an inflatable distal cuff 254, which forms a part of the airway device at a distal end 256 of the airway device. As shown in FIG. 22, the airway device 64f is placed in the patient's oral cavity 34 and the oropharyngeal area 36, with the proximal end 252 located at the patient's mouth 28, and the distal cuff 254 located in the oropharyngeal area, whereafter the distal cuff is inflated to form an air-blockage seal in the oropharyngeal area.

The airway device 64f can be entirely straight or entirely curved from the proximal end 252 thereof to the distal end 256 thereof, or can be formed with a straight section 258 having a prescribed length from the proximal end 252 toward, but not extending to, the distal end 256, with the remainder of the airway device, which extends to the distal end of the airway device, being formed as a curved section 260, which is integrally joined with the straight section at a juncture 262 thereof, all without departing from the spirit and scope of the invention.

Occasionally, a perfect air seal may not be formed by the inflated cuff 254, and air may leak around the seal, and undesirably result in air backflow through the patient's mouth 28.

In accordance with certain principles of the invention, an inflatable proximal cuff 264 is positioned on an intermediate portion of the tube 250, at a location which is a prescribed proximal distance from the proximal end 252 of the tube, where, upon placement of the airway device 64f as shown in FIG. 22, the proximal cuff is located in the patient's oral cavity 34. If the backflow of air through the patient's mouth 28 is detected after the distal cuff 254 has been inflated, the proximal cuff 264 will be inflated to provide an air seal in the oral cavity 34, and preclude any undesirable air backflow through the patient's mouth 28.

In another aspect of the invention, the tube 250 may be extended axially outward at the proximal end 258, by a prescribed extension distance, to form a proximal portion 266 of the airway device 64f. When the airway device 64f is placed in the patient's oral cavity and oropharyngeal area, as shown in FIG. 22, the proximal portion 266 will extend outward from the patient's mouth 28, in the same manner described above with respect to the airway device 64 (FIG. 10). This outward extension of the proximal portion from the patient's mouth 28 facilitates use of a face mask 268, which can be assembled over the proximal portion and placed over the patient's mouth to selectively form an air-blockage.

In general, the above-identified embodiments are not to be construed as limiting the breadth of the present invention. Modifications, and other alternative constructions, will be apparent which are with the spirit and scope of the invention as defined in the appended claims.

What is claimed is:

1. An airway device for ventilation of a patient having a mouth, an oral cavity, an oropharyngeal area, a glottic opening and vocal cords, an esophagus, an esophageal opening, a nasal passage and an inner nasal passage opening, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube; and
the tube being formed with a prescribed length, which extends from the proximal end of the tube to the distal end of the tube, where the prescribed length facilitates the locating of the distal end at the glottic opening and the vocal cords when the tube is positioned in the oral cavity and the oropharyngeal area.

2. An airway device for ventilation of a patient having a mouth, an oral cavity, an oropharyngeal area, a glottic opening and vocal cords, an esophagus, an esophageal opening, a nasal passage and an inner nasal passage opening, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube; and
the tube being formed with a proximal portion at the proximal end of the tube, where the proximal portion extends outward from the patient's mouth by a prescribed distance, when the tube is positioned in the oral cavity and the oropharyngeal area of the patient and the distal end is located at the glottic opening.

3. The airway device as set forth in claim 2, which further comprises:
the tube being formed with a prescribed length, which extends from the proximal end of the tube to the distal end of the tube, where the prescribed length facilitates the locating of the distal end at the glottic opening and the vocal cords of when the tube is positioned in the oral cavity and the oropharyngeal area.

4. The airway device as set forth in claim 1, which further comprises:
the tube formed with a straight section, which includes the proximal end and the proximal opening of the tube;
the tube formed with a curved section, which includes the distal end and the distal opening of the tube; and
the straight section joined with the curved section at a juncture thereof intermediate the proximal end and the distal end of the tube.

5. The airway device as set forth in claim 4, which further comprises:
the joined straight section and the curved section being formed in a hook-shaped configuration.

6. The airway device as set forth in claim 4, which further comprises:
the joined straight section and the curved section being formed in a J-shaped configuration.

7. The airway device as set forth in claim 4, which further comprises:
the curved section being formed in a generally quarter-circle configuration.

8. The airway device as set forth in claim 1, which further comprises:
an inflatable distal cuff mounted on the tube adjacent the distal end thereof at a location for forming a seal around the exterior of the tube and over the glottic opening to preclude communication between the oropharyngeal area and the glottic opening when the distal end is located at the glottic opening and the distal cuff is inflated.

9. The airway device as set forth in claim 1, which further comprises:
an inflatable distal cuff mounted on the tube adjacent the distal end thereof at a location for forming a seal over the esophageal opening to preclude communication between the esophagus and the oropharyngeal area when the distal end is located at the glottic opening and the distal cuff is inflated.

10. The airway device as set forth in claim 1, which further comprises:
an inflatable distal cuff mounted on the tube adjacent the distal end thereof which displaces and holds the epiglottis in a non-obstructing location spaced from the tube, when the distal cuff is inflated.

11. The airway device as set forth in claim 1, which further comprises:
an inflatable distal cuff mounted on the tube adjacent the distal end thereof which provides a centralizing adjustment of the distal end of the tube to locate the distal opening of the tube with respect to the glottic opening, when the distal cuff is inflated.

12. The airway device as set forth in claim 1, which further comprises:
an inflatable distal cuff mounted on the tube adjacent the distal end thereof which provides a surrounding and strengthening support for the distal end of the tube, when the distal cuff is inflated.

13. The airway device as set forth in claim 8, which further comprises:
the inflatable distal cuff mounted on the tube adjacent the distal end thereof at a location for forming a seal over the esophageal opening to preclude communication between the esophagus and the oropharyngeal area when the distal end is located at the glottic opening and the distal cuff is inflated.

14. The airway device as set forth in claim 8, which further comprises:
the inflatable distal cuff being located on the tube, inboard from the distal opening of the tube, by a distance which will prevent any portion of the cuff from obstructing any portion of the distal opening when the distal cuff is inflated.

15. The airway device as set forth in claim 1, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated.

16. The airway device as set forth in claim 8, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated.

17. The airway device as set forth in claim 13, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated.

18. The airway device as set forth in claim 1, which further comprises:
an inflatable intermediate cuff mounted at an intermediate location on the tube for forming a seal between the oropharyngeal area and the oral cavity to preclude communication between the oropharyngeal area and the mouth when the distal end is located at the glottic opening and the intermediate cuff is inflated.

19. The airway device as set forth in claim 8, which further comprises:
an inflatable intermediate cuff mounted at an intermediate location on the tube for forming a seal between the oropharyngeal area and the oral cavity to preclude communication between the oropharyngeal area and the oral cavity when the distal end is located at the glottic opening and the intermediate cuff is inflated.

20. The airway device as set forth in claim 13, which further comprises:
an inflatable intermediate cuff mounted at an intermediate location on the tube for forming a seal between the oropharyngeal area and the oral cavity to preclude communication between the oropharyngeal area and the oral cavity when the distal end is located at the glottic opening and the intermediate cuff is inflated.

21. The airway device as set forth in claim 15, which further comprises:
an inflatable intermediate cuff mounted at an intermediate location on the tube for forming a seal between the oropharyngeal area and the oral cavity to preclude communication between the oropharyngeal area and the oral cavity when the distal end is located at the glottic opening and the intermediate cuff is inflated.

22. The airway device as set forth in claim 1, which further comprises:
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

23. The airway device as set forth in claim 8, which further comprises:
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

24. The airway device as set forth in claim 13, which further comprises:
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

25. The airway device as set forth in claim 15, which further comprises:
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

26. The airway device as set forth in claim 8, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated; and
an inflatable intermediate cuff mounted at an intermediate location on the tube for forming a seal between the oropharyngeal area and the oral cavity to preclude communication between the oropharyngeal area and the mouth when the distal end is located at the glottic opening and the intermediate cuff is inflated.

27. The airway device as set forth in claim 8, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated; and
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

28. The airway device as set forth in claim 27, which further comprises:
the inflatable intermediate cuff mounted at the intermediate location on the tube for forming a seal between the oropharyngeal area and the oral cavity to preclude communication between the oropharyngeal area and the mouth when the distal end is located at the glottic opening and the intermediate cuff is inflated.

29. The airway device as set forth in claim 12, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated; and
an inflatable intermediate cuff mounted at an intermediate location on the tube for forming a seal between the oropharyngeal area and the oral cavity to preclude communication between the oropharyngeal area and the mouth when the distal end is located at the glottic opening and the intermediate cuff is inflated.

30. The airway device as set forth in claim 12, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated; and
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

31. The airway device as set forth in claim 30, which further comprises:
the inflatable intermediate cuff mounted at the intermediate location on the tube for forming a seal between the oropharyngeal area and the oral cavity to preclude communication between the oropharyngeal area and the mouth when the distal end is located at the glottic opening and the intermediate cuff is inflated.

32. The airway device as set forth in claim 13, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated; and
an inflatable intermediate cuff mounted at an intermediate location on the tube for forming a seal between the oropharyngeal area and the oral cavity to preclude communication between the oropharyngeal area and the mouth when the distal end is located at the glottic opening and the intermediate cuff is inflated.

33. The airway device as set forth in claim 13, which further comprises:
an inflatable proximal cuff mounted on the tube adjacent the proximal end thereof, and inboard from the proximal opening of the tube by a prescribed proximal distance for forming a seal in the oral cavity to preclude communication between the oropharyngeal area and the mouth when the distal end is located at the glottic opening and the proximal cuff is inflated; and
an inflatable intermediate cuff mounted at an intermediate location on the tube for forming a seal over the inner nasal passage opening to preclude communication between the oropharyngeal area and the nasal passage when the distal end is located at the glottic opening and the intermediate cuff is inflated.

34. The airway device as set forth in claim 33, which further comprises:
the inflatable intermediate cuff mounted at the intermediate location on the tube for forming a seal between the oropharyngeal area and the oral cavity to preclude communication between the oropharyngeal area and the mouth when the distal end is located at the glottic opening and the intermediate cuff is inflated.

35. The airway device as set forth in claim 2, which further comprises:
a face mask located on the proximal portion of the tube.

36. The airway device as set forth in claim 2, which further comprises:
the tube being formed by:
a hollow contoured element;
a hollow straight element having a distal section for assembly with the contoured element,
the hollow straight element having a proximal section extending outward from the hollow contoured element forming the proximal portion of the airway device, when the hollow straight element is assembled with the hollow contoured element.

37. The airway device as set forth in claim 36, which further comprises:
the hollow contoured element and the hollow straight element structured for a friction-fit assembly.

38. The airway device as set forth in claim 36, which further comprises:
the hollow contoured element formed with a hollow passage having a groove formed in the hollow passage, with the groove having an entry opening at one end of the hollow passage and extending to a base of the groove at a location within the passage;
the hollow straight element formed with a body, including the distal section, with the distal section being sized to be placed into the hollow passage of the hollow contoured element;
the hollow straight element formed with a projection extending radially outward from an outer surface of the distal section of the body; and
the projection being sized to fit into the entry opening of the groove, and to be moved within, and to the base of, the groove, when the hollow straight element is assembled with the hollow contoured element.

39. The airway device as set forth in claim 38, which further comprises:
means formed at the base of the groove for precluding movement by the hollow straight element, including the projection, axially from assembly within the hollow passage of the hollow contoured element.

40. The airway device as set forth in claim 2, which further comprises:
the tube being formed by:
a hollow contoured element;
a first hollow straight element having a distal section for assembly with the hollow contoured element;
a second hollow straight element having a distal section for assembly with the first hollow straight element; and
a proximal section of the first hollow straight element and a proximal section of the second hollow straight element forming the proximal portion of the tube when the hollow contoured element, the first straight element and the second straight element are assembled.

41. The airway device as set forth in claims 40 which further comprises:
the hollow contoured element being formed with a hollow passage which extends from one end of the hollow contoured element to at least an intermediate portion of the hollow contoured element;
the hollow passage of the hollow contoured element being formed with a prescribed internal diameter; and
the first hollow straight element being formed with a body including the distal section having an exterior diameter which is less that the prescribed internal diameter of the hollow passage of the hollow contoured element.

42. The airway device as set forth in claim 41, which further comprises:
the first hollow straight section being formed with the body having a proximal section which is joined with the distal section of the body;
the body of the first hollow straight element being formed with a hollow passage which extends from one end of the body into at least the proximal section of the body; and
the hollow passage of the first hollow straight element being formed with the prescribed internal diameter.

43. The airway device as set forth in claim 42, which further comprises:
the second hollow straight section being formed with a body including the distal section having an exterior diameter which is less that the prescribed internal diameter of the hollow passage of the first hollow straight element.

44. The airway device as set forth in claim 43, which further comprises:
the exterior diameter of the distal section of the body of the first hollow straight element is less than the prescribed internal diameter of the hollow passage of the hollow contoured element to facilitate assembling the distal section of the body of the first hollow straight element within the hollow passage of the hollow contoured element by friction fit; and
the exterior diameter of the distal section of the body of the second hollow straight element is less than the prescribed internal diameter of the hollow passage of the body of the first hollow straight element to facilitate assembling the distal section of the body of the second hollow straight element within the hollow passage of the body of the first hollow straight element by friction fit.

45. The airway device as set forth in claim 43, which further comprises:
the hollow passage of the body of the hollow contoured element having a contoured-element groove formed in the hollow passage, with the contoured-element groove having an entry opening at the one end of the hollow passage and extending to a base of the contoured-element groove at an intermediate location within the hollow passage;
the first hollow straight element formed with a first-element projection extending radially outward from an outer surface of the distal section of the body of the first hollow straight element; and
the first-element projection being sized to fit into the entry opening of the contoured-element groove, and to be moved within the contoured-element groove to the base of the groove, when the first hollow straight element is assembled with the hollow contoured element.

46. The airway device as set forth in claim 45, which further comprises:
means formed at the base of the contoured-element groove for precluding movement by the first hollow straight element, including the first-element projection, axially from assembly within the hollow passage of the hollow contoured element.

47. The airway device as set forth in claim 43, which further comprises:
the hollow passage of the body of the first hollow straight element having a first-element groove formed in the hollow passage, with the first-element groove having an entry opening at the one end of the hollow passage and extending to a base of the first-element groove at an intermediate location within the hollow passage;
the second hollow straight element formed with a second-element projection extending radially outward from an outer surface of the distal section of the body of the second hollow straight element; and
the second-element projection being sized to fit into the entry opening of the first-element groove, and to be moved within the first-element groove to the base of the first-element groove, when the second hollow straight element is assembled with the first hollow straight element.

48. The airway device as set forth in claim 47, which further comprises:
means formed at the base of the first-element groove for precluding movement by the second hollow straight element, including the second-element projection, axially from assembly within the hollow passage of the body of the first hollow straight element.

49. The airway device as set forth in claim 4, wherein the straight section of the tube is formed about a central axis, which further comprises:
an anterior distal surface formed at the distal end of the tube, contiguous with the distal opening of the tube; and
the anterior distal surface formed at an angle, within a range of more than zero degrees and less than ninety degrees, from a plane which is parallel to, and spaced from, the central axis of the straight section of the tube.

50. The airway device as set forth in claim 49, which further comprises:
the angle of the anterior distal surface is thirty degrees.

51. The airway device as set forth in claim 49, which further comprises:
the angle of the anterior distal surface is forty-five degrees.

52. The airway device as set forth in claim 4, wherein the straight section of the tube is formed about a central axis, which further comprises:
a posterior distal surface formed at the distal end of the tube, contiguous with the distal opening of the tube; and
the posterior distal surface formed at an angle, within a range of more than zero degrees and forty-five degrees, from a plane which is parallel to, and spaced from, the central axis of the straight section of the tube.

53. The airway device as set forth in claim 52, which further comprises:
the angle of the posterior distal surface is thirty degrees.

54. The airway device as set forth in claim 52, which further comprises:
the angle of the posterior distal surface is forty-five degrees.

55. The airway device as set forth in claim 1, wherein the tube is the airway device is an airway tube, which further comprises:
the passage formed through the airway tube with a prescribed diameter for receiving an endotracheal tube having an exterior diameter which is less than the prescribed diameter, where the endotracheal tube can be moved guidingly through the passage, with a distal end of the endotracheal tube extending from the distal end of the airway tube, through the glottic opening and into the trachea.

56. The airway device as set forth in claim 55, which further comprises:
a stylette positionable through the passage of the airway tube prior to moving the endotracheal tube through the passage; and
the stylette having a length which allows a distal portion of the stylette to extend from the distal end of the airway tube, through the glottic opening and into the trachea, and a proximal portion to extend from the proximal end of the airway tube outwardly from the mouth;
whereafter the endotracheal tube can be moved guidingly over the stylette through the glottic opening and into the trachea.

57. The airway device as set forth in claim 56, which further comprises:
the airway tube being removable from within the oropharyngeal area, the oral cavity and the mouth, and from the stylette, prior to the guiding movement of the endotracheal tube over the stylette.

58. An airway device for ventilation of a patient, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube; and
the tube being formed in a hook-shaped configuration.

59. An airway device for ventilation of a patient, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube; and
the tube being formed in a J-shaped configuration.

60. An airway device for ventilation of a patient, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube;
the tube being formed with a straight section and a curved section which are joined integrally at a juncture thereof; and
the curved section being formed in a generally quarter-round configuration.

61. An airway device for ventilation of a patient, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube;
the tube being formed with a proximal portion at the proximal end of the tube; and
the tube being further formed by:
a hollow contoured element; and
a hollow straight element having a distal section, for assembly with the contoured element, and a proximal section, which forms the proximal portion of the tube when the hollow straight element is assembled with the hollow contoured element.

62. The airway device as set forth in claim 61, which further comprises:
the hollow contoured element and the hollow straight element structured for a friction-fit assembly.

63. The airway device as set forth in claim 61, which further comprises:
the hollow contoured element formed with a hollow passage having a groove formed in the hollow passage, with the groove having an entry opening at one end of the hollow passage and extending to a base of the groove at an intermediate location within the passage;
the hollow straight element formed with a body having a distal section which is sized to be placed into the hollow passage of the hollow contoured element;
the hollow straight element formed with a projection extending radially outward from an outer surface of the body of the hollow straight element; and
the projection being sized to fit into the entry opening of the groove, and to be moved within, and to the base of, the groove, when the hollow straight element is assembled with the hollow contoured element.

64. The airway device as set forth in claim 63, which further comprises:
means formed at the base of the groove for precluding movement by the hollow straight element, including the projection, axially from assembly within the hollow passage of the hollow contoured element.

65. An airway device for ventilation of a patient, which comprises:
a tube formed with a proximal opening at a proximal end of the tube, and a distal opening at a distal end of the tube;
a passage formed through the tube from the proximal end to the distal end of the tube, with the passage being in communication with the proximal opening and the distal opening of the tube;
the tube being formed with a proximal portion at the proximal end of the tube; and
the tube being formed by:
a hollow contoured element;
a first hollow straight element having a distal section for assembly with the hollow contoured element;
a second hollow straight element having a distal section for assembly with the first hollow straight element; and
a proximal section of the body of the first hollow straight element, and a proximal section of the second hollow straight element forming the proximal portion of the tube when the hollow contoured element, the first straight element and the second straight element are assembled.

66. The airway device as set forth in claims 65 which further comprises:
the hollow contoured element being formed with a hollow passage which extends from one end of the hollow contoured element to at least an intermediate portion of the hollow contoured element;
the hollow passage of the hollow contoured element being formed with a prescribed internal diameter; and
the first hollow straight element being formed with a body having a distal section having an exterior diameter which is less that the prescribed internal diameter of the hollow passage of the hollow contoured element.

67. The airway device as set forth in claim 66, which further comprises:
the first hollow straight section being formed with the body having a proximal section which is joined with the distal section of the body;
the body of the first hollow straight element being formed with a hollow passage which extends from a proximal end of the body into at least the proximal section of the body;
the hollow passage of the first hollow straight element being formed with the prescribed internal diameter.

68. The airway device as set forth in claim 67, which further comprises:
the second hollow straight section being formed with a body having a distal section which is formed with an exterior diameter which is less that the prescribed interior diameter of the hollow passage of the first hollow straight element.

69. The airway device as set forth in claim 68, which further comprised:
the exterior diameter of the distal section of the body of the first hollow straight element is less than the prescribed internal diameter of the hollow passage of the hollow contoured element to facilitate assembling the distal section of the body of the first hollow straight element within the hollow passage of the hollow contoured element by friction fit; and
the exterior diameter of the distal section of the body of the second hollow straight element is less than the prescribed internal diameter of the hollow passage of the body of the first hollow straight element to facilitate assembling the distal section of the body of the second hollow straight element within the hollow passage of the body of the first hollow straight element by friction fit.

70. The airway device as set forth in claim 68, which further comprises:
the hollow passage of the body of the hollow contoured element having a contoured-element groove formed in the hollow passage, with the contoured-element groove having an entry opening at the one end of the hollow passage and extending to a base of the contoured-element groove at an intermediate location within the hollow passage;
the first hollow straight element formed with a first-element projection extending radially outward from an outer surface of the distal section of the body of the first hollow straight element; and
the first-element projection being sized to fit into the entry opening of the contoured-element groove, and to be moved within the contoured-element groove to the base of the groove, when the first hollow straight element is assembled with the hollow contoured element.

71. The airway device as set forth in claim 70, which further comprises:
means formed at the base of the contoured-element groove for precluding movement by the first hollow straight element, including the first-element projection, axially from assembly within the hollow passage of the hollow contoured element.

72. The airway device as set forth in claim 68, which further comprises:
the hollow passage of the body of the first hollow straight element having a first-element groove formed in the hollow passage, with the first-element groove having an entry opening at the one end of the hollow passage and extending to a base of the first-element groove at an intermediate location within the hollow passage;
the second hollow straight element formed with a second-element projection extending radially outward from an outer surface of the distal section of the body of the second hollow straight element; and
the second-element projection being sized to fit into the entry opening of the first-element groove, and to be moved within the first-element groove to the base of the first-element groove, when the second hollow straight element is assembled with the first hollow straight element.

73. The airway device as set forth in claim 72, which further comprises:
means formed at the base of the first-element groove for precluding movement by the second hollow straight element, including the second-element projection, axially from assembly within the hollow passage of the hollow contoured element.

74. An airway device for ventilation of a patient, which comprises:
a tube having a straight section, formed about a central axis, and a curved section;
the tube formed with a proximal opening at a proximal end of the tube, and a distal opening at a distal end of the tube;
a passage formed through the tube from the proximal end to the distal end of the tube, with the passage being in communication with the proximal opening and the distal opening of the tube;
an anterior distal surface formed at the distal end of the tube, contiguous with the distal opening of the tube; and
the anterior distal surface formed at an angle, within a range of more than zero degrees and less than ninety degrees, from a plane which is parallel to, and spaced from, the central axis of the straight section of the tube.

75. The airway device as set forth in claim 74, which further comprises:
the angle of the anterior distal surface is thirty degrees.

76. The airway device as set forth in claim 74, which further comprises:
the angle of the anterior distal surface is forty-five degrees.

77. An airway device for ventilation of a patient, which comprises:
a tube formed with a straight section, about a central axis, and a curved section;
the tube formed with a proximal opening at a proximal end of the tube, and a distal opening at a distal end of the tube;
a passage formed through the tube from the proximal end to the distal end of the tube, with the passage being in communication with the proximal opening and the distal opening of the tube;
a posterior distal surface formed at the distal end of the tube, contiguous with the distal opening of the tube; and
the posterior distal surface formed at an angle, within a range of more than zero degrees and forty-five degrees, from a plane which is parallel to, and spaced from, the central axis of the straight section of the tube.

78. The airway device as set forth in claim 77, which further comprises:
the angle of the posterior distal surface is thirty degrees.

79. The airway device as set forth in claim 77, which further comprises:
the angle of the posterior distal surface is forty-five degrees.

80. A method of using an airway device for ventilation of a patient having a mouth, an oral cavity, an oropharyngeal area, a glottic opening and vocal cords, an esophagus, an esophageal opening, a nasal passage and an inner nasal passage opening, which comprises the steps of:
forming a tube with a proximal opening at a proximal end of the tube, and a distal opening at a distal end of the tube;
forming a passage through the tube from the proximal end to the distal end of the tube, with the passage being in communication with the proximal opening and the distal opening of the tube;
forming the tube with a prescribed length, which extends from the proximal end of the tube to the distal end of the tube;
positioning the tube through the mouth and in the oral cavity and the oropharyngeal area; and
locating the distal end of the tube at the glottic opening and the vocal cords.

81. A method of using an airway device for ventilation of a patient having a mouth, an oral cavity, an oropharyngeal area, a glottic opening and vocal cords, an esophagus, an esophageal opening, a nasal passage and an inner nasal passage opening, which comprises the steps of:
forming a tube with a proximal opening at a proximal end of the tube, and a distal opening at a distal end of the tube;
forming a passage through the tube from the proximal end to the distal end of the tube, with the passage being in communication with the proximal opening and the distal opening of the tube;
forming the tube with a proximal portion at the proximal end of the tube;
forming the tube with a prescribed length, which extends from the proximal end of the tube, including the proximal portion, to the distal end of the tube;
positioning the tube through the mouth and in the oral cavity and the oropharyngeal area; and
retaining the proximal portion of the tube in a position extending outward from the patient's mouth for a prescribed distance, when the tube is positioned in the oral cavity and the oropharyngeal area of the patient.

82. The method as set forth in claim 81, which further comprises the step of:
placing a face mask onto the proximal portion of the tube.

83. The method as set forth in claim 81, which further comprises the step of:
positioning the distal end of the tube at the glottic opening and the vocal cords.

84. The method as set forth in claim 80, which further comprises the step of:
forming a seal around the exterior of the tube and over the glottic opening to preclude communication between the oropharyngeal area and the glottic opening when the distal end is located at the glottic opening.

85. The method as set forth in claim 80, which further comprises the step of:
forming a seal over the esophageal opening to preclude communication between the esophagus and the oropharyngeal area when the distal end is located at the glottic opening.

86. The method as set forth in claim 80, which further comprises the step of:
holding the epiglottis in a non-obstructing location spaced from the tube, when the distal end of the tube is located at the glottic opening.

87. The method as set forth in claim 80, which further comprises the step of:
providing a centralizing adjustment of the distal end of the tube to locate the distal opening of the tube in general alignment with the glottic opening, when the distal end of the tube is at the glottic opening.

88. The method as set forth in claim 80, which further comprises the step of:
providing a surrounding and strengthening support for the distal end of the tube, when the distal end is at the glottic opening.

89. The method as set forth in claim 80, which further comprises the step of:
forming a seal in the oral cavity to preclude communication between the oropharyngeal area and the mouth when the distal end is located at the glottic opening.

90. The method as set forth in claim 80, which further comprises the step of:
forming a seal between the oropharyngeal area and the oral cavity to preclude communication between the oropharyngeal area and the oral cavity when the distal end is located at the glottic opening.

91. The method as set forth in claim 80, which further comprises the step of:
forming a seal over the inner nasal passage opening to preclude communication between the oropharyngeal area and the nasal passage when the distal end is located at the glottic opening.

92. A method of making an airway device for ventilation of a patient, which comprises the steps of:
forming a contoured element;
forming a hollow passage in at least a portion of the contoured element at a proximal end thereof;
forming a straight element having a proximal section and a distal section; and
assembling the distal section of the straight element within the hollow passage of the contoured element, with the proximal section of the straight element forming a proximal portion of the airway device, which extends from the proximal end of the contoured element, when the straight element is in assembly with the contoured element.

93. The method as set forth in claim 92, which further comprises the step of:
securing by friction fit, as an assembly, the contoured element and the straight element.

94. The method as set forth in claim 92, which further comprises the step of:
forming a groove in the hollow passage of the contoured element to a base of the groove with the base being located an intermediate location within the hollow passage;
forming an entry opening in the groove at the proximal end of the contoured element;
forming the distal section of the straight element for placement into the hollow passage of the contoured element;
forming a projection extending radially outward from an outer surface of the distal section of the straight element; and
sizing the projection to fit into the entry-opening of the groove, and to be moved within the groove to the base thereof, when the straight element is assembled with the contoured element.

95. The method as set forth in claim 94, which further comprises the step of:
forming a means at the base of the groove for precluding relative movement of the straight element, including the projection, axially from assembly with the contoured element.

96. The method as set forth in claim 92, wherein the straight element is a first straight element, which further comprises the steps of:
forming a hollow passage in the proximal section of the first straight element;
forming a second straight element having a proximal section and a distal section; and
assembling a distal section of the second straight element within the hollow passage of the first straight element to form an assembly of the first straight element and the second straight element, with the proximal section of the first straight element and the proximal section of the second straight element forming the proximal portion of the airway device, which extends from the proximal end of the contoured element.

97. The method as set forth in claim 80, which further comprises the steps of:
forming the tube integrally with a straight section, which includes the proximal end of the tube, and a curved section, which includes the distal end of the tube;
forming an anterior distal surface at the distal end of the tube, contiguous with the distal opening of the tube; and
forming the anterior distal surface at an angle, within a range of more than zero degrees and less than ninety degrees, from a plane which is parallel to, and spaced from, a central axis of the straight section of the tube.

98. The method as set forth in claim 97, which further comprises the step of:
forming the anterior distal surface at an angle of thirty degrees.

99. The method as set forth in claim 97, which further comprises the step of:
forming the anterior distal surface at an angle of forty-five degrees.

100. The method as set forth in claim 80, which further comprises the steps of:
forming the tube integrally with a straight section, which includes the proximal end of the tube, and a curved section, which includes the distal end of the tube;
forming a posterior distal surface at the distal end of the tube, contiguous with the distal opening of the tube; and
forming the posterior distal surface at an angle, within a range of more than zero degrees and forty-five degrees, from a plane which is parallel to, and spaced from, a central axis of the straight section of the tube.

101. The method as set forth in claim 100, which further comprises the step of:
forming the posterior distal surface at an angle of thirty degrees.

102. The method as set forth in claim 97, which further comprises the step of:
forming the posterior distal surface at an angle of forty-five degrees.

103. The method as set forth in claim 80, wherein the tube is an airway tube, which further comprises the step of:
forming the passage through the airway tube with a prescribed diameter for receiving an endotracheal tube having an exterior diameter which is less than the prescribed diameter, where the endotracheal tube can be moved guidingly through the passage, with a distal end of the endotracheal tube extending from the distal end of the airway tube, through the glottic opening and into the trachea.

104. The method as set forth in claim 103, which further comprises the steps of:
positioning a stylette through the passage of the airway tube prior to moving the endotracheal tube through the passage; and
forming the stylette with a length which allows a distal portion of the stylette to extend from the distal end of the airway tube, through the glottic opening and into the trachea, and a proximal portion of the stylette to extend from the proximal end of the airway tube outwardly from the mouth.

105. The method as set forth in claim 104, which further comprises the step of:
moving the endotracheal tube guidingly over the stylette with the distal end thereof being moved through the glottic opening and into the trachea.

106. The method as set forth in claim 105, which further comprises the step of:
removing the airway tube from within the oropharyngeal area, the oral cavity and the mouth, and from about the stylette, prior to the guiding movement of the endotracheal tube over the stylette.

107. An airway device for ventilation of a patient having a mouth, an oral cavity and an oropharyngeal area, which comprises:
a distal end;
a tube having a proximal end;
an inflatable distal cuff at the distal end which, when the airway device is placed in the patient's oral cavity and the oropharyngeal area, is locatable in the oropharyngeal area to form an air-blockage seal in the oropharyngeal area upon inflation of the distal cuff;
the tube extending from the proximal end thereof to the distal cuff; and
an inflatable proximal cuff positioned on an intermediate portion of the tube, a prescribed proximal distance from the proximal end of the tube, to form an air-blockage seal in the patient's oral cavity, when the proximal cuff is inflated.

108. An airway device for ventilation of a patient having a mouth, an oral cavity and an oropharyngeal area, which comprises:
a distal end;
a tube having a proximal end;
an inflatable distal cuff at the distal end which, when the airway device is placed in the patient's oral cavity and the oropharyngeal area, is locatable in the oropharyngeal area to form an air-blockage seal in the oropharyngeal area upon inflation of the distal cuff;
the tube extending from the proximal end thereof to the distal cuff; and
a proximal portion formed as an outward axial extension, which extends by a prescribed extension distance from the proximal end of the tube, where the proximal portion extends outward from the patient's mouth when the airway device is placed in the patient's oral cavity and oropharyngeal area, to facilitate use of a face mask with the airway device.

109. The airway device as set forth in claim 108, which further comprises:
an inflatable proximal cuff positioned on an intermediate portion of the tube, a prescribed proximal distance from the proximal end of the tube, to form an air-blockage seal in the patient's oral cavity, when the proximal cuff is inflated.
